(19) United States
(12) Patent Application Publication
(10) Pub. No.: US 2025/0201346 A1
(43) Pub. Date: Jun. 19, 2025

(54) USING MACHINE LEARNING MODELS FOR DETECTING MINIMUM RESIDUAL DISEASE (MRD) IN A SUBJECT

(71) Applicant: Illumina, Inc., San Diego, CA (US)

(72) Inventors: Seyedmohammadjafar Hashemidoulabi, Lakewood, CO (US); Sven Bilke, Lemon Grove, CA (US); James Han, San Carlos, CA (US); Yunjiao Zhu, San Diego, CA (US); Anindita Dutta, San Francisco, CA (US); Fan Song, San Diego, CA (US)

(21) Appl. No.: 18/984,697

(22) Filed: Dec. 17, 2024

Related U.S. Application Data
(60) Provisional application No. 63/559,375, filed on Feb. 29, 2024.
(60) Provisional application No. 63/611,660, filed on Dec. 18, 2023.

(51) Int. Cl. G16B 40/00; C12Q 1/6886; G16B 20/20

(52) U.S. Cl. CPC G16B 40/00; C12Q 1/6886; G16B 20/20

(57) ABSTRACT

This disclosure describes methods, non-transitory computer-readable media, and systems that detect minimal residual disease (MRD) within a sample of interest. For example, in some cases, the disclosed systems identify, for an initial genomic sample of a subject infected with cancer, a tumor fingerprint comprising variants at a target genomic region. The disclosed systems further determine, for a sample of interest of the subject, a set of sample of interest nucleotide reads associated with the target genomic region. The disclosed system process the set of sample of interest nucleotide reads using a first machine learning model and process panel of normals nucleotide reads using one or more additional machine learning models. The disclosed systems compare scores determined from the outputs of the machine learning models to predict whether the sample of interest has minimal residual disease related to the cancer.

### CROSS-REFERENCE TO RELATED APPLICATIONS

This application claims priority to and the benefit of U.S. Provisional Patent Application No. 63/559,375, entitled, “USING MACHINE LEARNING MODELS FOR DETECTING MINIMUM RESIDUAL DISEASE (MRD) IN A SUBJECT,” filed on Feb. 29, 2024 (IP-2751-PRV2) and U.S. Provisional Patent Application No. 63/611,660, entitled, “USING MACHINE LEARNING MODELS FOR DETECTING MINIMUM RESIDUAL DISEASE (MRD) IN A SUBJECT,” filed on Dec. 18, 2023 (IP-2751-PRV). Both of the aforementioned applications is hereby incorporated by reference in its entirety.

### BACKGROUND

In recent years, biotechnology firms and research institutions have improved hardware and software platforms for determining various characteristics of a genomic sample or other nucleic-acid polymer. For instance, platforms have been developed for analyzing a nucleotide sequence from a sample of interest (e.g., a genomic sample) and identifying the nucleobases contained therein-such as by using a sequencing process performed via conventional Sanger sequencing or sequencing-by-synthesis (SBS). Through such a process, existing platforms can create one or more nucleotide reads that indicate the sequence of nucleobases contained in the nucleotide sequence. Some existing platforms use these nucleotide reads to make further determinations about the sample of interest. For example, some existing systems use the nucleotide reads to detect minimal residual disease (MRD—-the presence of residual cancer cell—-in post-treatment cancer patients.

Despite these advances, existing MRD detection systems suffer from technical shortcomings that result in inflexible and inaccurate operation. For instance, many existing systems are inflexible in that they fail to accommodate errors in the read data generated during sequencing when performing MRD detection. Indeed, some existing systems directly analyze the nucleotide reads generated from a genomic sample to detect the presence of circulating tumor DNA (ctDNA) within the sample. By detecting ctDNA, these existing systems may generate a positive result for MRD. Sequencing imperfections, however, can lead to noisy nucleotide reads having erroneous base call sequences that resemble ctDNA. Existing systems often fail to flexibly distinguish between these erroneous reads and actual ctDNA supporting reads. In other words, existing systems typically fail to implement methods and models that produce MRD detection results that have taken noisy nucleotide reads into account.

Additionally, existing MRD detection systems are also inaccurate. In particular, existing systems often provide inaccurate detection results. Indeed, by failing to implement methods and models that accommodate noisy nucleotide reads, existing systems often provide detection results that have been influenced by such noisy reads. To illustrate, by failing to distinguish between noisy reads that resemble ctDNA and actual ctDNA supporting reads, existing systems may provide a false positive result for MRD based on such noisy reads.

These along with additional problems and issues exist with regard to existing MRD detection systems.

### SUMMARY

Embodiments of the present disclosure provide benefits and/or solve one or more of the foregoing or other problems in the art with systems, methods, and non-transitory computer-readable media that use a unique and sophisticated combination of machine learning models to detect MRD in post-treatment cancer patients. In particular, the disclosed systems combine whole genome sequencing (WGS) and machine learning to create a comprehensive solution for detecting ctDNA in genomic samples. To illustrate, the disclosed systems can use WGS and an initial sample from a cancer patient (e.g., a tumor sample obtained via biopsy) to create a fingerprint that uniquely corresponds to the presence of the cancer in the patent. The disclosed systems use the fingerprint in training and implementing a collection of machine learning models for MRD detection. In particular, after treatment, the disclosed systems can implement the collection of machine learning models to score a plurality of additional samples, including a post-treatment sample taken from the patient and one or more samples from a panel of normals. Based on the generated scores, the disclosed systems can determine whether MRD has been detected. In this manner, the disclosed systems flexibly accommodate noisy nucleotide reads for accurate MRD detection.

Additional features and advantages of one or more embodiments of the present disclosure are outlined in the description which follows, and in part can be determined from the description, or may be learned by the practice of such example embodiments.

### BRIEF DESCRIPTION OF THE DRAWINGS

This disclosure will describe one or more embodiments of the invention with additional specificity and detail by referencing the accompanying figures. The following paragraphs briefly describe those figures, in which:

FIG. 1 illustrates a schematic diagram of a computing system in which an MRD detection system can operate in accordance with one or more embodiments.

FIG. 2 illustrates an overview diagram of the MRD detection system performing MRD detection using various samples in accordance with one or more embodiments.

FIG. 3 illustrates the MRD detection system determining a tumor fingerprint for a subject of interest in accordance with one or more embodiments.

FIG. 4 illustrates the MRD detection system determining nucleotide reads with respect to a tumor fingerprint for a subject in accordance with one or more embodiments.

FIG. 5 illustrates the MRD detection system generating a class 1 dataset in accordance with one or more embodiments.

FIG. 6 illustrates the MRD detection system generating class 0 datasets and training a collection of machine learning models for MRD detection in accordance with one or more embodiments.

FIG. 7 illustrates the MRD detection system using a collection of machine learning models to process various samples to test a subject of interest for MRD in accordance with one or more embodiments.

FIG. 8 illustrates graphs reflecting experimental results regarding the effectiveness of the MRD detection system in accordance with one or more embodiments.

FIG. 9 illustrates a flowchart of a series of acts for detecting minimal residual disease within a sample of interest in accordance with one or more embodiments.

FIG. 10 illustrates a block diagram of an example computing device in accordance with one or more embodiments.

### DETAILED DESCRIPTION

One or more embodiments described herein include a minimal residual disease (MRD) detection system that combines whole genome sequencing (WGS) with a unique combination of machine learning models to provide a comprehensive solution for detecting MRD in genomic samples. To illustrate, in one or more embodiments, the MRD detection system uses WGS to create a unique fingerprint for an initial sample (e.g., a tumor sample) taken from a cancer patient. After treatment, the MRD detection system can use a collection of machine learning models to detect MRD within the patient by comparing a post-treatment sample (e.g., a plasma sample) taken from the patient to additional samples from a panel of normals. In some cases, the MRD detection system identifies paired-end reads to test from each sample with respect to the unique fingerprint created from the initial sample. In some embodiments, the MRD detection system further personalizes the training of the machine learning models using training data determined with respect to the unique fingerprint. Thus, the MRD detection system can personalize the analysis to the unique combination of mutations associated with the specific cancer in the particular patient.

To illustrate, in one or more embodiments, the MRD detection system identifies, for an initial genomic sample of a subject infected with a type of cancer, a tumor fingerprint comprising variants at a target genomic region. Additionally, the MRD detection system determines, for a sample of interest corresponding to a subsequent genomic sample of the subject, a sample of interest set of nucleotide reads associated with the target genomic region. The MRD detection system further processes extracted features from the sample of interest set of nucleotide reads to generate a sample of interest score using a first machine learning model that was trained with sample of interest training data. The MRD detection system also processes extracted features from panel of normals nucleotide reads associated with the target genomic region to generate one or more panel of normals scores using one or more additional machine learning models that were trained with panel of normals training data. The MRD detection system compares the sample of interest score to the one or more panel of normals scores to predict whether the sample of interest has minimal residual disease related to the type of cancer.

As mentioned above, in one or more embodiments, the MRD detection system performs MRD detection using WGS and various genomic samples. For instance, the MRD detection system can use samples from a subject of interest (e.g., a subject to be tested for MRD) and a plurality of other subjects. In some cases, the MRD detection system generates nucleotide reads from each sample via WGS and uses the nucleotide reads during the training and inference of the MRD detection process.

For example, in some cases, the MRD detection system uses WGS to generate one or more nucleotide reads for an initial genomic sample (e.g., a tumor sample) of the subject of interest. Using the nucleotide read(s), the MRD detection system can create a tumor fingerprint that includes or indicates variants at a target genomic region. Specifically, the MRD detection system creates a tumor fingerprint that is unique to the presence of the cancer in the subject of interest. The MRD detection system can use the tumor fingerprint for personalized training and inference.

To illustrate, as previously mentioned, in one or more embodiments, the MRD detection system also trains and implements a set of machine learning models for MRD detection. For instance, in some cases, the MRD detection system trains and implements a first machine learning model that corresponds to the subject of interest and one or more additional machine learning models that correspond to a panel of normals.

In some embodiments, the MRD detection system trains the machine learning models with respect to the tumor fingerprint for the subject of interest by using non-tumor nucleotide reads determined with respect to the tumor fingerprint. In particular, the MRD detection system can create a plurality of class 0 datasets that include paired-end reads generated from samples of the subject of interest and the panel of normals, where each paired-end read is from a pseudo fingerprint genomic position within its corresponding sample. In other words, the paired-end reads do not overlap with the target genomic region of the tumor fingerprint. In some cases, the MRD detection system also trains the machine learning models using a class 1 dataset that includes tumor supporting nucleotide reads generated from other tumor samples of other subjects, which include paired-end reads that overlap the unique fingerprint of their corresponding sample and support the tumor allele.

In one or more embodiments, the MRD detection system implements the machine learning models with respect to the tumor fingerprint for the subject of interest by using, from each sample to be tested, paired-end reads that do overlap the target genomic region of the tumor fingerprint. In certain embodiments, the MRD detection system tests the same samples that are used in creating the class 0 datasets.

In some embodiments, the MRD detection system implements the machine learning models by using the first machine learning model to analyze a subsequent genomic sample (e.g., a plasma sample) from the subject of interest. The MRD detection system further uses the one or more additional machine learning models to analyze additional samples from the panel of normals. The MRD detection system can use each machine learning model to generate a score for the corresponding sample. Further, the MRD detection system can compare the score from the first machine learning model to the score(s) from the additional machine learning model(s). Based on the comparison, the MRD detection system can predict whether the subsequent genomic sample includes minimal residual disease.

As indicated above, the MRD detection system provides several technical advantages over existing systems. For instance, the MRD detection system offers a new, unconventional approach to detecting MRD within post-treatment cancer patients by implementing a unique and sophisticated combination of machine learning models to analyze various samples from various subjects. Indeed, where existing systems may focus on the sequencing determined for a sample of interest to detect the presence of MRD-supporting circulating tumor DNA (ctDNA), the MRD detection system implements a plurality of machine learning models to compare the sample of interest to various other samples, including other tumor samples and samples from a panel of normals. To illustrate, the MRD detection system implements an unconventional ordered combination of steps that involves determining model inputs with respect to a unique fingerprint for the subject of interest, using the machine learning models to score each of the model inputs, and comparing the generated scores to determine whether MRD has been detected. The MRD detection system can further train the machine learning models using a unique combination of training data that personalizes the models to the subject of interest.

Based on this new approach to MRD detection, the MRD detection system provides more flexibility compared to existing systems. Indeed, by implementing a collection of machine learning models that compares a sample of interest to various other samples, the MRD detection system flexibly accommodates noisy nucleotide reads that may result from the sequencing process. In particular, the MRD detection system flexibly accounts for flaws in the sequencing process to better distinguish between erroneous reads that may resemble ctDNA and actual ctDNA supporting reads that would result in MRD detection. Further, by training and implementing the machine learning models with respect to the fingerprint determined for the subject of interest, the MRD detection system flexibly tailors the analysis of the machine learning models to the combination of mutations that are unique to the presence of the particular cancer in the subject of interest.

Beyond improved flexibility, the MRD detection system provides improved accuracy when compared to existing systems. In particular, the MRD detection system more accurately detects MRD within a sample of interest. Indeed, by implementing an approach that better distinguishes between nucleotide reads that merely resemble ctDNA and actual ctDNA supporting reads, the MRD detection system can more accurately determine when nucleotide reads from a sample of interest indicate MRD within the sample. For instance, by identifying actual ctDNA supporting reads with improved accuracy, the MRD detection system can reduce the false positives that may be generated under existing systems.

As suggested by the foregoing discussion, this disclosure utilizes a variety of terms to describe features and benefits of the MRD detection system. Additional detail is hereafter provided regarding the meaning of these terms as used in this disclosure. As used in this disclosure, for instance, the term “genomic sample” (or simply “sample”) refers to a target genome or portion of a genome undergoing an assay or sequencing. For example, a genomic sample includes one or more sequences of nucleotides isolated or extracted from a sample organism (or a copy of such an isolated or extracted sequence). In particular, a genomic sample includes a full genome that is isolated or extracted (in whole or in part) from a sample organism and composed of nitrogenous heterocyclic bases. A genomic sample can include a segment of deoxyribonucleic acid (DNA), ribonucleic acid (RNA), or other polymeric forms of nucleic acids or chimeric or hybrid forms of nucleic acids noted below. In some cases, the genomic sample is found in a sample prepared or isolated by a kit and received by a sequencing device. In some instances, a genomic sample more broadly refers to the substance (e.g., the biological matter) from which the one or more sequences of nucleotides are derived. In other words, in some implementations, a genomic sample includes the source of the nucleotide sequences that will undergo an assay or sequencing.

Relatedly, as used herein, the term “tumor sample” refers to a genomic sample taken from a tumor. For example, a tumor sample can include tissue extracted from a tumor via a biopsy or a target genome or portion of a target genome derived from such tissue. Additionally, as used herein, the term “blood sample” refers to a genomic sample taken from blood. For example, a blood sample can include a portion of blood extracted via a blood draw or a target genome or portion of a target genome derived from such blood. Similarly, as used herein, the term “plasma sample” can include a genomic sample taken from plasma. For example, a plasma sample can include at least a portion of the plasma component of blood that has been isolated via a centrifuge.

As used herein, the term “subject” refers to a sample organism from which a genomic sample is derived. In particular, a subject includes a sample organism from which a genomic sample is isolated or extracted. For example, in some cases, a subject includes a human subject, but a subject can include various other sample organisms in various implementations.

As used herein, the term “subject of interest” includes a subject to undergo testing for MRD. For instance, a subject of interest can include a subject that has been diagnosed with and/or treated for cancer and will undergo testing to determine whether minimal residual disease related to the cancer remains within the subject. Relatedly, as used herein, the term “sample of interest” refers to a genomic sample extracted, isolated, or otherwise derived from a subject of interest. In particular, a sample of interest refers to the genomic sample used to test the subject of interest for MRD. For instance, in some cases, the MRD detection system tests a sample of interest to identify actual ctDNA supporting reads that would result in MRD detection. In some instances, a sample of interest corresponds to a post-treatment genomic sample.

Also, as used herein, the term “nucleotide read” (or simply “read”) refers to an inferred or predicted sequence of one or more nucleobases (or nucleobase pairs) from all or part of a sample genomic sequence (e.g., a sample genomic sequence, complementary DNA). In particular, a nucleotide read includes a determined or predicted sequence of nucleobase calls for a nucleotide fragment (or group of monoclonal nucleotide fragments) from a sequencing library corresponding to a genomic sample. For example, in some embodiments, the MRD detection system determines a nucleotide read by generating nucleobase calls for nucleobases passed through a nanopore of a nucleotide-sample slide, determined via fluorescent tagging, or determined from a well in a flow cell. In some cases, a nucleotide read can refer to a particular type of read, such as a nucleotide read synthesized from sample library fragments that are shorter than a threshold number of nucleobases (e.g., SBS reads). In these or other cases, another type of nucleotide read can refer to (i) assembled nucleotide reads that have been assembled from shorter nucleotide reads to form a contiguous sequence (e.g., assembled nucleotide reads) satisfying a threshold number of nucleobases, (ii) circular consensus sequencing (CCS) reads satisfying the threshold number of nucleobases, or (iii) nanopore long reads satisfying the threshold number of nucleobases.

As used herein, the term “paired-end read” refers to a nucleotide read generated by sequencing both ends of a nucleotide fragment. In particular, a paired-end read refers to a nucleotide read that is composed of a pair of component reads-such as a forward read and a reverse read. Indeed, in some cases, a paired-end read includes a first read created via sequencing of the nucleotide fragment beginning at one end of the fragment and a second read created via sequencing beginning at the other end of the nucleotide fragment.

Additionally, as used herein, the term “variant” refers to a nucleobase or multiple nucleobases that do not align with, differs from, or varies from a corresponding nucleobase (or nucleobases) in a reference sequence or a reference genome. Examples a variant include, but is not limited to, a single nucleotide variant (SNF), a single nucleotide polymorphism (SNP), an indel, a phased somatic variant, a copy number variant (CNV), or a structural variant (SV) that indicates nucleobases in a sample nucleotide sequence that differ from nucleobases in corresponding genomic coordinates of a reference sequence or a reference genome.

Further, as used herein, the term “tumor fingerprint” (or simply “fingerprint”) refers to a set of variants associated with a subject diagnosed with a type of cancer. In particular, a tumor fingerprint refers to a set of variants that are uniquely associated with (e.g., linked to) the presence of the particular type of cancer in the particular subject. Indeed, in some instances, even where two subjects are infected with the same type of cancer, the set of variants linked to the type of cancer can differ in each subject due to the particular biological makeup of each subject. Thus, in some cases, a tumor fingerprint is unique to each subject. In one or more embodiments, a tumor fingerprint is located at a particular genomic region of the subject.

As used herein, the term “genomic coordinate” (or sometimes simply “coordinate”) refers to a particular location or position of a nucleobase within a genome (e.g., an organism's genome or a reference genome). In some cases, a genomic coordinate includes an identifier for a particular chromosome of a genome and an identifier for a position of a nucleobase within the particular chromosome. For instance, a genomic coordinate or coordinates may include a number, name, or other identifier for a somatic or sex chromosome (e.g., chr1 or chrX) and a particular position or positions, such as numbered positions following the identifier for a chromosome (e.g., chr1: 1234570 or chr1: 1234570-1234870). In certain implementations, a genomic coordinate refers to a source of a reference genome (e.g., mt for a mitochondrial DNA reference genome or SARS-COV-2 for a reference genome for the SARS-COV-2 virus) and a position of a nucleobase within the source for the reference genome (e.g., mt: 16568 or SARS-COV-2:29001). By contrast, in certain cases, a genomic coordinate refers to a position of a nucleobase within a reference genome without reference to a chromosome or source (e.g., 29727).

Additionally, as used herein, a “genomic region” refers to a range of genomic coordinates. Like genomic coordinates, in certain implementations, a genomic region may be identified by an identifier for a chromosome and a particular position or positions, such as numbered positions following the identifier for a chromosome (e.g., chr1: 1234570-1234870).

As used herein, the term “fingerprint of interest” refers to a tumor fingerprint associated with the subject of interest. For instance, in some cases, the MRD detection system determines a fingerprint of interest based on sequencing a tumor sample from the subject of interest. Additionally, as used herein, the term “target genomic region” refers to a genomic region associated with the fingerprint of interest. In other words, a target genomic region can refer to a site of the fingerprint of interest within a genome of the subject of interest or a reference genome. Further, as used herein, the term “pseudo fingerprint genomic region” refers to a genomic region that does not overlap with the target genomic region associated with the fingerprint of interest.

As used herein, the term “tumor supporting nucleotide read” refers to a nucleotide read that indicates the presence of cancer within a subject. In particular, a tumor supporting nucleotide read refers to a nucleotide read (e.g., a paired-end read) that includes or has been identified as including one or more variants from the set of variants associated with the presence of cancer within a subject. For instance, in certain implementations, a tumor supporting nucleotide read includes a paired-end read corresponding to (e.g., generated from) a tumor sample of a subject and having a first read and a second read that overlap with a genomic region associated with a fingerprint for the tumor sample and support a tumor allele associated with the tumor sample. In some cases, a tumor supporting nucleotide read includes a nucleotide read identified (e.g., classified) by a machine learning model as indicating the presence of cancer within the subject from which the corresponding sample was extracted.

Additionally, as used herein, the term “non-tumor nucleotide read” refers to a nucleotide read that does not indicate the presence of cancer within a subject. In particular, a non-tumor nucleotide read includes a nucleotide read (e.g., a paired-end read) that does not include or has been identified as not including variants associated with the presence of cancer within a subject. For instance, in some embodiments, a non-tumor nucleotide read includes a paired-end read that does not overlap with the site of a fingerprint determined for a subject. In some cases, a non-tumor nucleotide read is generated from a sample of a particular subject and determined with respect to the same subject. In some implementations, however, a non-tumor nucleotide read is generated from the sample of one subject but determined with respect to another subject. For instance, as will be described in more detail below, in one or more embodiments, the MRD detection system uses a plurality of non-tumor nucleotide reads that correspond to the subject of interest and a plurality of additional subjects, where each non-tumor nucleotide read is located at a pseudo fingerprint genomic region. In some implementations, a non-tumor nucleotide read includes a nucleotide read identified (e.g., classified) by a machine learning model as not indicating the presence of cancer within the subject from which the corresponding sample was extracted.

As used herein, the term “sample of interest nucleotide read” refers to a nucleotide read generated from a sample of interest. In particular, a sample of interest nucleotide read includes a nucleotide read (e.g., a paired-end read) used to test the sample of interest for MRD. Indeed, in some cases, a sample of interest nucleotide read includes a nucleotide read created from a post-treatment sample of a subject of interest and used to detect MRD within the subject of interest. In some cases, a sample of interest nucleotide read includes a nucleotide read that overlaps the target genomic region of the fingerprint of interest.

Additionally, as used herein, the term “panel of normals” refers to a set of samples that establish a baseline. In particular, a panel of normals can include a set of normal samples that establish a baseline for MRD detection. To illustrate, a panel of normals can include a set of samples from various non-cancer subjects. In some implementations, the MRD detection system uses a panel of normals as data generated from the panel of normals should not be indicative of MRD (e.g., does not indicate the presence of ctDNA).

Further, as used herein, the term “panel of normals nucleotide read” refers to a nucleotide read generated from the panel of normals. In particular, a panel of normals nucleotide read includes a nucleotide read (e.g., a paired-end read) that corresponds to (e.g., is generated from) a sample from the panel of normals. In some cases, a panel of normals nucleotide read includes a nucleotide read used to test a corresponding sample from the panel of normals.

As used herein, the term “machine learning model” refers to a computer representation that can be tuned (e.g., trained) based on inputs to approximate unknown functions. In particular, the term “machine-learning model” can include a model that utilizes algorithms to learn from, and make predictions on, known data by analyzing the known data to learn to generate outputs that reflect patterns and attributes of the known data. For instance, a machine-learning model can include but is not limited to a neural network (e.g., a convolutional neural network, recurrent neural network or other deep learning network), a decision tree (e.g., a gradient boosted decision tree), association rule learning, inductive logic programming, support vector learning, Bayesian network, regression-based model (e.g., censored regression), principal component analysis, or a combination thereof.

As used herein, the term “machine learning model” refers to a computer algorithm or a collection of computer algorithms that can be tuned (e.g., trained) based on inputs to approximate unknown functions used for generating the corresponding outputs. In particular, a machine learning model can include one or more computer algorithms that automatically improve for a particular task through iterative outputs or predictions based on the use of data. For example, a machine learning model can utilize one or more learning techniques to improve accuracy and/or effectiveness. More specifically, a machine learning model can use algorithms to learn from, and make predictions on, known data by analyzing the known data to learn to generate outputs that reflect patterns and attributes of the known data. Example machine learning models include, but are not limited to, various types of neural networks (e.g., convolutional neural networks and recurrent neural networks, decision trees (e.g., gradient-boosting decision trees, such as XGBoost), support vector machines, linear regression models, and Bayesian networks.

As used herein, the term “training data” refers to data used to train a machine learning model to test a sample. In particular, training data can include data used to train a machine learning model to analyze nucleotide reads (e.g., paired-end reads) from a sample (e.g., analyze the nucleotide reads for indicators of MRD, such as the presence of ctDNA). As will be explained in more detail below, training data can include tumor supporting nucleotide reads and non-tumor nucleotide reads. Relatedly, as used herein, the term “sample of interest training data” refers to training data used to train a machine learning model to test a sample of interest. Similarly, as used herein, the term “panel of normals training data” refers to training data used to train a machine learning model to test a sample from a panel of normals.

Additionally, as used herein, the term “score” refers to a value determined from outputs generated by a machine learning model based on testing a sample. In particular, a score can include a value determined from outputs generated by a machine learning model that indicates whether MRD has been detected within the sample. For instance, a score can include a numerical score determined from outputs of a machine learning model for a particular sample, where the value of the score indicates the number of nucleotide reads (e.g., paired-end reads) from the sample that have been determined to indicate MRD (e.g., indicate the presence of ctDNA). Indeed, in some embodiments, a score includes a count of the nucleotide reads identified by a machine learning model as tumor supporting nucleotide reads. Relatedly, as used herein, the term “sample of interest score” refers to a score generated based on outputs of a machine learning model for a sample of interest. Similarly, as used herein, the term “panel of normals score” refers to a score generated based on outputs a machine learning model for a sample from a panel of normals.

As used herein, the term “class 1 dataset” refers to a set of training data that includes tumor supporting nucleotide reads. Additionally, as used herein, the term “class 0 dataset” refers to a set of training data that includes non-tumor nucleotide reads. In one or more embodiments, the sample of interest training data and the panel of normals training data each include training data from a class 1 dataset and one or more class 0 datasets. More detail regarding the class 1 dataset and class 0 datasets, their composition, and their use in training machine learning models will be provided below.

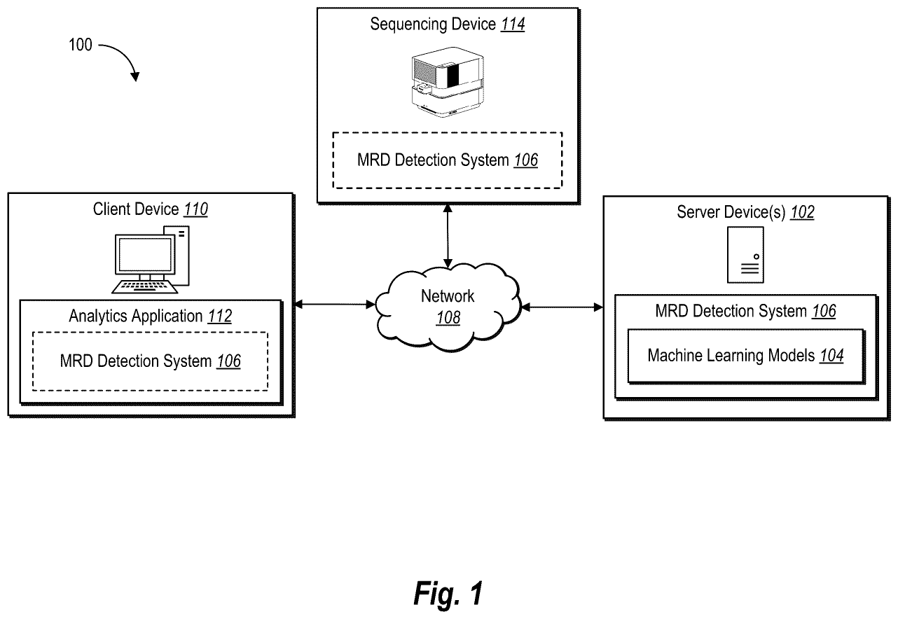

The following paragraphs describe the MRD detection system with respect to illustrative figures that portray example embodiments and implementations. FIG. 1 illustrates a schematic diagram of a computing system 100 in which a MRD detection system 106 operates in accordance with one or more embodiments. As illustrated, the computing system 100 includes one or more server device(s) 102 connected to a client device 110 and a sequencing device 114 via a network 108. While FIG. 1 shows one embodiment of the MRD detection system 106, this disclosure describes alternative embodiments and configurations below.

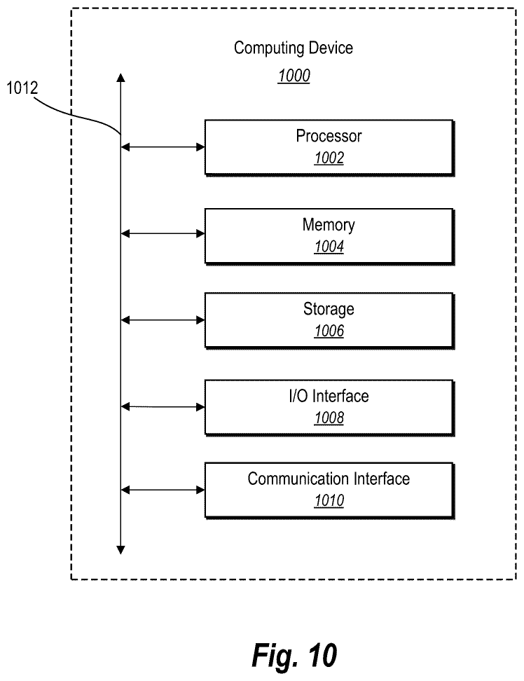

As shown in FIG. 1, the server device(s) 102, the client device 110, and the sequencing device 114 are connected via the network 108. Accordingly, each of the components of the computing system 100 can communicate via the network 108. The network 108 comprises any suitable network over which computing devices can communicate. Example networks are discussed in additional detail below with respect to FIG. 10.

As indicated by FIG. 1, the sequencing device 114 comprises a computing device for sequencing a genomic sample or other nucleic-acid polymer. In some embodiments, the sequencing device 114 analyzes nucleotide fragments or oligonucleotides extracted from genomic samples to generate nucleotide reads or other data utilizing computer-implemented methods and systems either directly or indirectly on the sequencing device 114. More particularly, the sequencing device 114 receives nucleotide-sample slides (e.g., flow cells) comprising nucleotide fragments (e.g., genomic DNA fragments) extracted from samples and further copies and determines the nucleobase sequence of such extracted nucleotide fragments.

In addition, or in the alternative to communicating across the network 108, in some embodiments, the sequencing device 114 bypasses the network 108 and communicates directly with the server device(s) 102 or the client device 110. Additionally, as shown in FIG. 1, in one or more embodiments, the sequencing device 114 includes the MRD detection system 106.

As further indicated by FIG. 1, the server device(s) 102 may generate, receive, analyze, store, and transmit digital data, such as data for amino-acid sequences or nucleotide sequences. As shown in FIG. 1, the sequencing device 114 may send (and the server device(s) 102 may receive) various data from the sequencing device 114, including data representing amino-acid sequences or nucleotide sequences (e.g., base calls and/or corresponding quality scores). The server device(s) 102 may also communicate with the client device 110. In particular, the server device(s) 102 can send data representing amino-acid sequences or nucleotide sequences (e.g., base calls and/or corresponding quality scores) to the client device 110.

Additionally, as shown in FIG. 1, the server device(s) 102 can include the MRD detection system 106. In one or more embodiments, as explained further below, the MRD detection system 106 analyzes nucleotide reads generated from various samples-including a sample of interest and one or more samples from a panel of normal—-to detect MRD. To illustrate, the MRD detection system 106 can use machine learning models 104 to score each of the samples for MRD based on their corresponding nucleotide reads. In particular, the MRD detection system 106 can use a first machine learning model to generate a sample of interest score for the sample of interest based on a set of sample of interest nucleotide reads (e.g., features extracted from the corresponding nucleotide reads). Further, the MRD detection system 106 can use one or more additional machine learning models to generate one or more panel of normals scores for one or more samples from the panel of normals based on corresponding panel of normals nucleotide reads. Based on the generated scores, the MRD detection system 106 can determine whether MRD has been detected for the sample of interest.

In some embodiments, the server device(s) 102 comprise a distributed collection of servers where the server device(s) 102 include a number of server devices distributed across the network 108 and located in the same or different physical locations. Further, the server device(s) 102 can comprise a content server, an application server, a communication server, a web-hosting server, or another type of server.

In some cases, the server device(s) 102 is located at or near a same physical location of the sequencing device 114 or remotely from the sequencing device 114. Indeed, in some embodiments, the server device(s) 102 and the sequencing device 114 are integrated into a same computing device. The server device(s) 102 may run software on the sequencing device 114 or the MRD detection system 106 to generate, receive, analyze, store, and transmit digital data, such as by sending or receiving scores generated for samples and/or MRD detection results. In some embodiments, the sequencing device 114 or the MRD detection system 106 store and access a database or table of scores generated for samples and/or MRD detection results.

As further illustrated and indicated in FIG. 1, the client device 110 can generate, store, receive, and send digital data. In particular, the client device 110 can receive data for amino-acid sequences or nucleotide sequences (or variants thereof), scores generated for various samples, or MRD detection results determined for a sample of interest from the server device(s) 102 and/or the sequencing device 114. The client device 110 can accordingly present data concerning the amino-acid sequences or nucleotide sequences (or variants thereof), the scores generated for the various samples, or the MRD detection results determined with respect to the sample of interest within a graphical user interface to a user associated with the client device 110.

The client device 110 illustrated in FIG. 1 may comprise one of various types of client devices. For example, in some embodiments, the client device 110 includes a non-mobile device, such as a desktop computer or server, or another type of client device. In yet other embodiments, the client device 110 includes a mobile device, such as a laptop, a tablet, a mobile telephone, or a smartphone. Additional details with regard to the client device 110 are discussed below with respect to FIG. 10.

As further illustrated in FIG. 1, the client device 110 includes an analytics application 112. The analytics application 112 may be a web application or a native application stored and executed on the client device 110 (e.g., a mobile application, desktop application). The analytics application 112 can include instructions that (when executed) cause the client device 110 to receive data from the MRD detection system 106 and present data from the sequencing device 114 and/or the server device(s) 102. Furthermore, the analytics application 112 can instruct the client device 110 to display data for scores generated for various samples or MRD detection results determined with respect to a sample of interest.

As further illustrated in FIG. 1, the MRD detection system 106 may be located on the client device 110 as part of the analytics application 112 or on the sequencing device 114. Accordingly, in some cases, the MRD detection system 106 is implemented by (e.g., located entirely or in part) on the client device 110. As mentioned, in yet other cases, the MRD detection system 106 is implemented by one or more other components of the computing system 100, such as the sequencing device 114. In particular, the MRD detection system 106 can be implemented in a variety of different ways across the server device(s) 102, the network 108, the client device 110, and the sequencing device 114.

Though FIG. 1 illustrates the components of the computing system 100 communicating via the network 108, in some cases, the components of the computing system 100 can also communicate directly with each other, bypassing the network 108. For instance, and as previously mentioned, in some implementations, the client device 110 communicates directly with the sequencing device 114. Additionally, in some embodiments, the client device 110 communicates directly with the MRD detection system 106. Moreover, the MRD detection system 106 can access one or more databases housed on or accessed by the server device(s) 102 or elsewhere in the computing system 100.

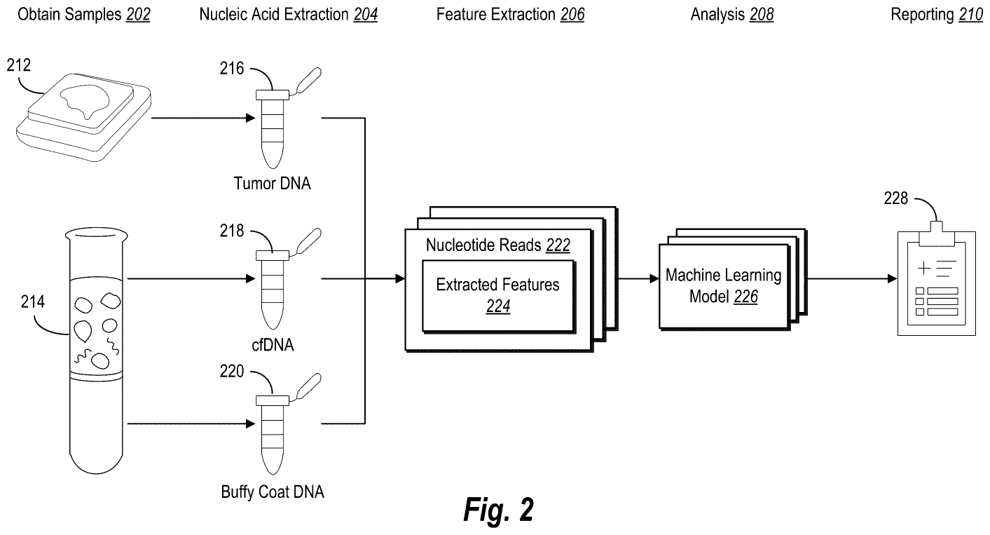

As discussed above, the MRD detection system 106 can analyze a sample of interest from a subject of interest, as well as one or more additional samples from one or more additional subjects, to determine whether the sample of interest has MRD. FIG. 2 illustrates an overview diagram of the MRD detection system 106 performing MRD detection using various samples in accordance with one or more embodiments.

As shown in FIG. 2, the MRD detection system 106 performs MRD detection by performing acts within a process that involves obtaining samples 202, nucleic acid extraction 204, feature extraction 206, analysis 208, and reporting 210.

Indeed, as shown in FIG. 2, the MRD detection system 106 can perform MRD detection using various types of samples. For instance, as shown, the MRD detection system 106 can perform MRD detection using one or more tumor samples, such as the tumor sample 212. In particular, the tumor sample 212 can include a sample obtained from a tumor (e.g., via a biopsy of the tumor) of a subject infected with a type of cancer. In some embodiments, the tumor sample 212 includes a formalin-fixed paraffin (FFP) sample.

As will be explained below, the MRD detection system 106 can use the one or more tumor samples to train and implement the collection of machine learning models that are used to perform MRD detection. For instance, the MRD detection system 106 can use a tumor sample from the subject of interest (e.g., as an initial genomic sample) to establish a tumor fingerprint for the subject of interest. Further, the MRD detection system 106 can use one or more additional tumor samples from one or more additional subjects.

As further shown in FIG. 2, the MRD detection system 106 can perform MRD detection using one or more blood samples, such as the blood sample 214. In particular, the blood sample 214 can be obtained via a blood draw from a subject. In some instances, the MRD detection system 106 uses one or more individual components of the blood sample 214 (e.g., obtained via centrifuging the blood sample 214). Indeed, the MRD detection system 106 can use one or more of a plasma sample, a buffy coat sample, or a sample of red blood cells from the blood sample 214.

As will be explained below, the MRD detection system 106 can use the one or more blood samples (e.g., or individual components of the blood sample(s)) to train and implement the collection of machine learning models that are used to perform MRD detection. For instance, in some cases, the MRD detection system 106 can use a plasma sample from the subject of interest (e.g., as a sample of interest) that will be tested for MRD. Further, the MRD detection system 106 can use one or more additional samples from a panel of normals.

As further illustrated in FIG. 2, the MRD detection system 106 uses DNA extracted from the samples obtained. For instance, the MRD detection system 106 uses tumor DNA 216 extracted from the tumor sample 212. Further, the MRD detection system 106 uses cell-free DNA (cfDNA) 218 and/or buffy coat DNA 220 extracted from the blood sample 214.

Additionally, as illustrated, the MRD detection system 106 generates nucleotide reads (e.g., the nucleotide reads 222) from the extracted DNA. For instance, in some cases, the MRD detection system 106 generates multiple nucleotide reads per sample. In one or more embodiments, the MRD detection system 106 generates the nucleotide reads via WGS. Further, in some embodiments, the MRD detection system 106 generates the nucleotide reads by generating paired-end reads. Indeed, the MRD detection system 106 can generate a plurality of paired-end reads for each sample.

As FIG. 2 also shows, the MRD detection system 106 can determine extracted features for the nucleotide reads (e.g., the extracted features 224 for the nucleotide reads 222). For instance, as will be explained more below, the MRD detection system 106 can extract a plurality of features from each nucleotide read (e.g., from each paired-end read). In some cases, the MRD detection system 106 analyzes the extracted features to perform MRD detection.

Indeed, as further illustrated, the MRD detection system 106 uses a collection of machine learning models (e.g., the machine learning model 226) to analyze the nucleotide reads (e.g., the extracted features). For instance, as will be explained in more detail below, the MRD detection system 106 can use a first machine learning model to analyze the sample of interest (e.g., analyze sample of interest nucleotide reads) and can use one or more additional machine learning models to analyze one or more additional samples from the panel of normals (e.g., analyze panel of normals nucleotide reads).

As shown, based on the analysis, the MRD detection system 106 generates a detection result 228. In particular, the MRD detection system 106 determines whether the sample of interest has MRD. In one or more embodiments, the MRD detection system 106 provides the detection result 228 (e.g., an indication of whether MRD has been detected) for display within a graphical user interface of a computing device.

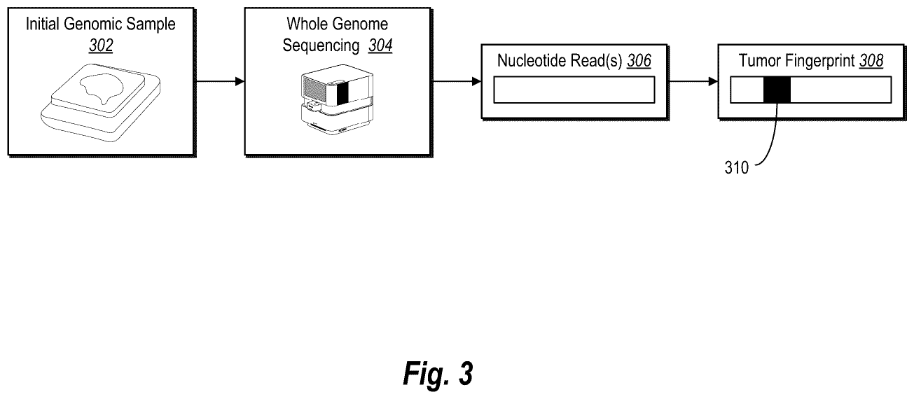

As described above, the MRD detection system 106 can perform MRD detection using one or more tumor samples. For instance, the MRD detection system 106 can use a tumor sample from a subject of interest to determine a tumor fingerprint for the subject of interest. The MRD detection system 106 can further perform the MRD detection with respect to the tumor fingerprint. FIG. 3 illustrates the MRD detection system 106 determining a tumor fingerprint for a subject of interest in accordance with one or more embodiments.

As shown in FIG. 3, the MRD detection system 106 performs whole genome sequencing 304 on an initial genomic sample 302 (e.g., a tumor sample) from a subject of interest. In particular, the MRD detection system 106 can perform the whole genome sequencing 304 on tumor DNA extracted from the initial genomic sample 302 as described above with reference to FIG. 2. Based on performing the whole genome sequencing 304, the MRD detection system 106 generates one or more nucleotide reads 306 for the initial genomic sample 302. For example, the MRD detection system 106 can generate one or more paired-end reads via the sequencing 304.

As further shown in FIG. 3, the MRD detection system 106 can determine a tumor fingerprint 308 for the subject of interest (i.e., a fingerprint of interest) based on the one or more nucleotide reads 306 generated from the initial genomic sample 302. As indicated, in some embodiments, the MRD detection system 106 determines the tumor fingerprint 308 by identifying a target genomic region 310 having the variants that are uniquely associated with the presence of the type of cancer within the subject of interest.

To illustrate, in one or more embodiments, the MRD detection system 106 compares the one or more nucleotide reads 306 generated from the initial genomic sample 302 to a reference genome. For example, the MRD detection system 106 can align the one or more nucleotide reads 306 with the reference genome and compare the one or more nucleotide reads 306 to the reference genome based on the alignment. Based on the comparison, the MRD detection system 106 can determine which nucleobases within the one or more nucleotide reads 306 represent variants. Thus, the MRD detection system 106 can determine a set of variants from the one or more nucleotide reads 306, establishing the tumor fingerprint 308 at the target genomic region 310.

By establishing the tumor fingerprint 308 for the subject of interest, the MRD detection system 106 establishes a baseline for what the presence of cancer looks like in the subject of interest. In particular, the MRD detection system 106 establishes a link between the particular type of cancer and the infected subject of interest on a nucleic-acid level (e.g., establishes the nucleic-acid indicators). Further, the MRD detection system 106 identifies the location of this link within the genome of the subject of interest with respect to the reference genome. Thus, as will be described further below, the MRD detection system 106 can use the tumor fingerprint 308 and the target genomic region 310 associated with the tumor fingerprint 308 to guide the MRD detection process.

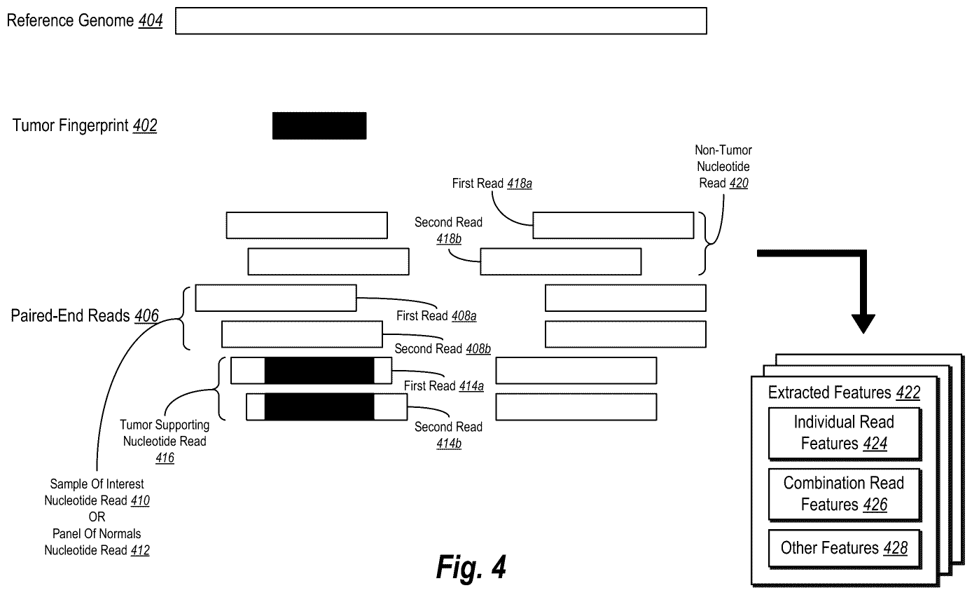

Indeed, as previously mentioned, in one or more embodiments, the MRD detection system 106 performs MRD detection with respect to a tumor fingerprint determined for a subject of interest (i.e., a fingerprint of interest). For instance, in some embodiments, the MRD detection system 106 determines one or more sets of nucleotide reads for use in training and implementing a collection of machine learning models for MRD detection with respect to the tumor fingerprint determined for the subject of interest. In some cases, the MRD detection system 106 further determines one or more sets of nucleotide reads with respect to tumor fingerprints of other subjects. FIG. 4 illustrates the MRD detection system 106 determining nucleotide reads with respect to a tumor fingerprint for a subject in accordance with one or more embodiments.

As shown in FIG. 4, the MRD detection system 106 determines a tumor fingerprint 402 for a subject. As indicated, the MRD detection system 106 can determine the tumor fingerprint 402 with respect to a reference genome 404, such as by aligning nucleotide reads generated from a sample of the subject using the reference genome 404. As further shown, the MRD detection system 106 identifies the genomic region associated with the tumor fingerprint 402. Indeed, by determining the tumor fingerprint 402 using the reference genome 404, the MRD detection system 106 also determines the location of the tumor fingerprint 402 within the genome of the subject. Though FIG. 4 illustrates a single genomic region for the tumor fingerprint 402, the MRD detection system 106 can identify multiple genomic regions for the tumor fingerprint 402 in certain implementations. Indeed, in some cases, the MRD detection system 106 identifies a repeating pattern of the set of variants associated with the tumor fingerprint 402.

As further shown in FIG. 4, the MRD detection system 106 determines a plurality of paired-end reads 406 and aligns the plurality of paired-end reads 406 with respect to the reference genome 404. In one or more embodiments, the plurality of paired-end reads 406 include paired-end reads generated from the sample from which the tumor fingerprint 402 was determined. In some embodiments, the plurality of paired-end reads 406 include paired-end reads generated from a different sample from the sample from which the tumor fingerprint 402 was determined. In some cases, the plurality of paired-end reads 406 include paired-end reads from a different subject than the subject for which the tumor fingerprint 402 was determined. Though FIG. 4 illustrates the MRD detection system 106 using paired-end reads, it should be noted that the MRD detection system 106 can use single-end reads in various implementations.

As illustrated by FIG. 4, the MRD detection system 106 can use the alignment of the plurality of paired-end reads 406 with respect to the reference genome 404 to identify paired-end reads for use in training and implementing a collection of machine learning models for MRD detection. For instance, in some cases, the MRD detection system 106 identifies paired-end reads that overlap with the tumor fingerprint 402 (e.g., overlap with the genomic region associated with the tumor fingerprint 402). In some instances, the MRD detection system 106 identifies paired-end reads that overlap with the tumor fingerprint 402 and support the tumor allele (e.g., include the set of variants of the tumor fingerprint 402). In some implementations, the MRD detection system 106 identifies paired-end reads that do not overlap with the tumor fingerprint 402.

For instance, FIG. 4 illustrates a paired-end read that includes a first read 408a and a second read 408b that overlap the tumor fingerprint 402. In other words, the first read 408a and the second read 408b overlap with the genomic region associated with the tumor fingerprint 402. As indicated, the MRD detection system 106 can use the paired-end read as a sample of interest nucleotide read 410 or a panel of normals nucleotide read 412. For instance, FIG. 4 shows that the first read 408a and the second read 408b do not support the tumor allele, or it is unknown if the first read 408a and the second read 408b support the tumor allele. Thus, in some instances, the MRD detection system 106 uses the paired-end read as a sample of interest nucleotide read in determining whether a corresponding sample of interest has MRD.

As another example, FIG. 4 illustrates another paired-end read that includes a first read 414a and a second read 414b that overlaps the tumor fingerprint 402. In this instance, the first read 414a and the second read 414b are explicitly shown as supporting the tumor allele. For instance, the first read 414a and the second read 414b can include the set of variants associated with the tumor fingerprint 402. As indicated, the MRD detection system 106 can use the paired-end read as a tumor supporting nucleotide read 416 in certain embodiments. For instance, the MRD detection system 106 can use the paired-end read as part of a training dataset of tumor supporting nucleotide reads generated from various tumor samples of various subjects.

FIG. 4 further illustrates a paired-end read that includes a first read 418a and a second read 418b that do not overlap the tumor fingerprint 402. As indicated, the MRD detection system 106 can use the paired-end read as a non-tumor nucleotide read 420. For example, where the tumor fingerprint 402 includes a fingerprint of interest, the MRD detection system 106 can determine that the first read 418a and the second read 418b are located in a pseudo fingerprint genomic region. In one or more embodiments, the MRD detection system 106 uses the paired-end read as part of one or more training datasets of non-tumor nucleotide reads generated from the subject of interest and/or one or more samples from a panel of normals.

As further illustrated in FIG. 4, the MRD detection system 106 determines extracted features (e.g., the extracted features 422) from the determined nucleotide reads. For instance, in one or more embodiments, the MRD detection system 106 determines a set of extracted features for each paired-end read. As an example, FIG. 4 shows the extracted features 422 including individual read features 424, combination read features 426, and other features 428.

In one or more embodiments, the individual read features 424 include features extracted from the first read and the second read of a paired-end read individually. For instance, in some embodiments, the individual read features 424 include one or more of MapQ scores for each read, average base call quality scores for each read, position of mismatch on each read, mismatch base quality for each read, the number of soft-clipped bases for each read, the number of hard-clipped bases for each read, the base values at the genomic region of the fingerprint (where applicable) for each read as well as one base to the left and one base to the right, total number of mismatches compared to a reference genome (e.g., HG38), the mean of base call qualities of mismatched bases for each read, the standard deviation (std) of base qualities of mismatched bases for each read, the template length (insert size) for each read, the reference length, or the reads themselves (referred to as R1 or R2 for the first or second read, respectively).

Additionally, in one or more embodiments, the combination read features 426 include features extracted from the first read and the second read of a paired-end read together. In other words, the MRD detection system 106 analyzes the paired-end read as a whole by analyzing the combination of the first read and the second read to determine the combination read features 426. In some embodiments, the combination read features 426 include one or more of the length of overlap between the first read and the second read, or the total number of mismatches between the first read and the second read in the overlapping region.

Further, in one or more embodiments, the other features 428 include various additional features determined by the MRD detection system 106. For instance, in some cases, the other features 428 include one or more of counts of the A, C, T, and G nucleobases at the genomic region associated with the tumor fingerprint 40—-where applicable-across the pile-up (e.g., a total count for each nucleobase across all reads overlapping the genomic region), the allele represented by the reference genome 404, and the tumor allele used in determining which paired-end reads to use (e.g., the tumor allele associated with or represented by the tumor fingerprint 402).

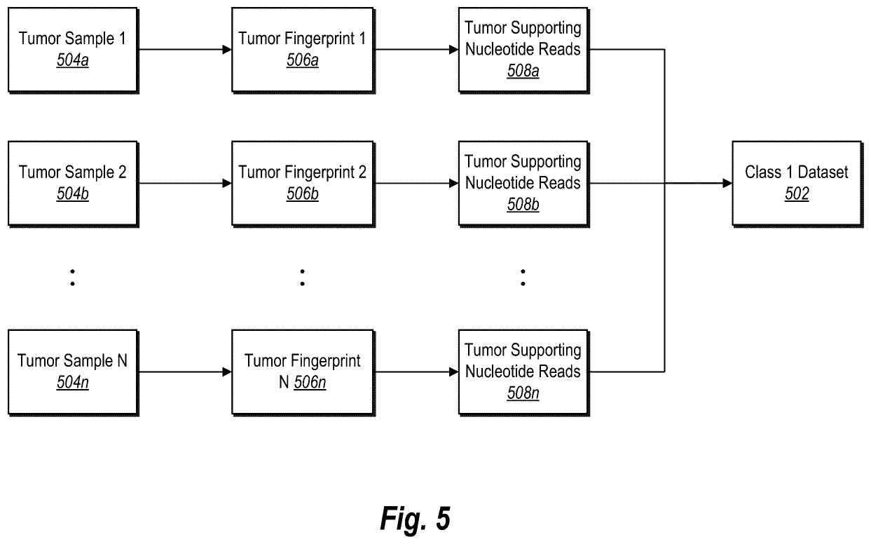

As previously mentioned, the MRD detection system 106 uses nucleotide reads in training a collection of machine learning models for MRD detection. In particular, the MRD detection system 106 creates various datasets that include different types of nucleotide reads and uses the datasets in training the machine learning models. For instance, in one or more embodiments, the MRD detection system 106 generates and uses a class 1 dataset that includes tumor supporting nucleotide reads. FIG. 5 illustrates the MRD detection system 106 generating a class 1 dataset in accordance with one or more embodiments.

As shown in FIG. 5, the MRD detection system 106 generates a class 1 dataset 502 using a plurality of tumor samples 504a-504n. Each tumor sample from the plurality of tumor samples 504a-504n can include a sample obtained from a corresponding subject infected with a type of cancer (e.g., via a biopsy of the tumor). To be clear, the plurality of tumor samples 504a-504n can include samples taken from subjects other than the subject of interest that will be tested for MRD. In one or more embodiments, each tumor sample is obtained from a different subject. In some embodiments, each tumor sample includes a FFP sample.

In some cases, the type of cancer associated with each tumor sample from the plurality of tumor samples 504a-504n is the same type of cancer as the type of cancer infecting the subject of interest that will be tested for MRD. In certain implementations, however the type of cancer associated with one or more of the tumor samples is a different type of cancer than the type of cancer infecting the subject of interest. Further, in some instances the type of cancer differs among the plurality of tumor samples 504a-504n. In some embodiments, however, each tumor sample from the plurality of tumor samples 504a-504n can include the same type of cancer.

As further shown in FIG. 5, the MRD detection system 106 determines a plurality of tumor fingerprints 506a-506n for the plurality of tumor samples 504a-504n. In particular, the MRD detection system 106 determines a tumor fingerprint for each of the tumor samples 504a-504n. For instance, the MRD detection system 106 determines a tumor fingerprint 506a for the tumor sample 504a, a tumor fingerprint 506b for the tumor sample 504b, and a tumor fingerprint 506n for the tumor sample 504n.

In one or more embodiments, the MRD detection system 106 determines a tumor fingerprint for a tumor sample as discussed above with reference to FIG. 3. Indeed, the MRD detection system 106 can perform whole genome sequencing on the tumor sample (e.g., on tumor DNA extracted from the tumor sample) to generate one or more nucleotide reads for the tumor sample. The MRD detection system 106 can further analyze the one or more nucleotide reads to determine the tumor fingerprint. In other words, the MRD detection system 106 can use the one or more nucleotide reads to identify the set of variants that are uniquely associated with (e.g., linked to) the presence of the particular type of cancer in the particular subject from which the tumor sample was obtained. By determining a tumor fingerprint for a tumor sample, the MRD detection system 106 can further determine the genomic region associated with the tumor fingerprint. In particular, the MRD detection system 106 can determine the genomic region of the tumor fingerprint in the genome of the subject with respect to the reference genome.

Additionally, as illustrated by FIG. 5, the MRD detection system 106 determines sets of tumor supporting nucleotide reads 508a-508n for the plurality of tumor samples 504a-504n. In particular, the MRD detection system 106 determines a set of tumor supporting nucleotide reads for each of the tumor samples 504a-504n. For instance, the MRD detection system 106 determines a set of tumor supporting nucleotide reads 508a for the tumor sample 504a, a set of tumor supporting nucleotide reads 508b for the tumor sample 504b, and a set of tumor supporting nucleotide reads 508n for the tumor sample 504n. In one or more embodiments, the MRD detection system 106 determines a set of tumor supporting nucleotide reads for a tumor sample using the tumor fingerprint created for the tumor sample.

To illustrate, to generate the set of tumor supporting nucleotide reads 508a for the tumor sample 504a, the MRD detection system 106 can generate a plurality of nucleotide reads based on the tumor sample 504a. In particular, the MRD detection system 106 can generate a plurality of paired-end reads based on the tumor sample 504a. In one or more embodiments, the MRD detection system 106 generates the plurality of paired-end reads via whole genome sequencing.

Further, the MRD detection system 106 can identify or select the set of tumor supporting nucleotide reads 508a from the plurality of paired-end reads generated for the tumor sample 504a. For instance, the MRD detection system 106 can analyze the plurality of paired-end reads generated for the tumor sample 504a to identify which paired-end reads are tumor supporting nucleotide reads. Indeed, the MRD detection system 106 can analyze the plurality of paired-end reads to identify paired-end reads that include a first read and a second read that overlap with the genomic region of the tumor fingerprint 506a and support the tumor allele associated with the tumor sample 504a. The MRD detection system 106 can include one or more of these paired-end reads within the set of tumor supporting nucleotide reads 508a for the tumor sample 504a.

Thus, the MRD detection system 106 can determine a set of tumor supporting nucleotide reads for each of the tumor samples 504a-504n. The MRD detection system 106 can include these sets of tumor supporting nucleotide reads within the class 1 dataset 502. As will be shown below, the MRD detection system 106 can share the class 1 dataset 502 among all machine learning models that will be trained and implemented for MRD detection. In other words, the MRD detection system 106 can train each of the machine learning models using the tumor supporting nucleotide reads included in the class 1 dataset 502. In one or more embodiments, the MRD detection system 106 includes a nucleotide read within the class 1 dataset 502 by including features extracted from the nucleotide read (e.g., as discussed above with reference to FIG. 4) within the class 1 dataset 502.

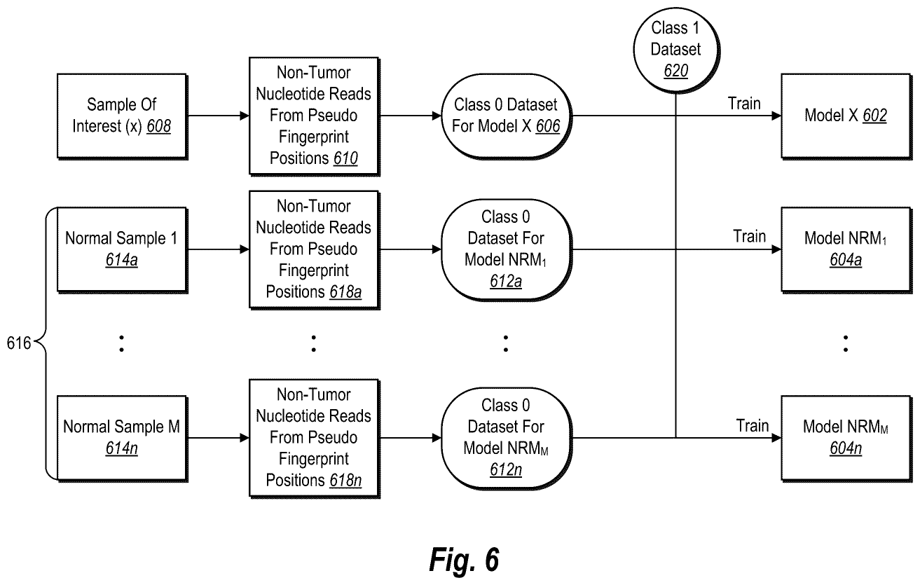

As previously discussed, in one or more embodiments, the MRD detection system 106 further generates and uses one or more class 0 datasets to train a collection of machine learning models for MRD detection. For instance, in certain implementations, the MRD detection system 106 generates and uses a class 0 dataset for each machine learning model. FIG. 6 illustrates the MRD detection system 106 generating and using a class 0 dataset for each machine learning model in accordance with one or more embodiments. FIG. 6 further illustrates the MRD detection system 106 training the machine learning models using a class 1 dataset and multiple class 0 datasets in accordance with one or more embodiments.

Indeed, as shown in FIG. 6, the MRD detection system 106 trains a first machine learning model 602 (labeled “Model X”) and additional machine learning models 604a-604n (labeled “Model NRM1” to “Model NRMM,” respectively). Though FIG. 6 illustrates a plurality of additional machine learning models, the MRD detection system 106 can use one additional machine learning model in certain implementations. In one or more embodiments, the first machine learning model 602 and the additional machine learning models 604a-604n each include a gradient-boosting decision tree, such as an XGBoost model. In some instances, the first machine learning model 602 and the additional machine learning models 604a-604n include a different type of machine learning model, such as a neural network.

As shown in FIG. 6, the MRD detection system 106 generates a class 0 dataset 606 for the first machine learning model 602 using a sample of interest 608. In one or more embodiments, the sample of interest 608 corresponds to a subsequent genomic sample taken from the subject of interest to be tested for MRD after an initial genomic sample taken from the subject of interest. For instance, in some embodiments, the sample of interest 608 includes a post-treatment sample obtained from the subject of interest. In some implementations, the sample of interest 608 includes a blood sample or, more specifically, a plasma sample taken from the subject of interest.

In one or more embodiments, the MRD detection system 106 generates the class 0 dataset 606 by determining non-tumor nucleotide reads 610 from the sample of interest 608. In one or more embodiments, the MRD detection system 106 determines the non-tumor nucleotide reads 610 from the sample of interest 608 using a tumor fingerprint associated with the sample of interest 608 (e.g., the tumor fingerprint determined for the subject of interest using a tumor sample from the subject of interest). Indeed, as indicated in FIG. 6, the non-tumor nucleotide reads 610 are at pseudo fingerprint genomic regions within the sample of interest 608. In other words, the non-tumor nucleotide reads 610 do not overlap with the target genomic region associated with the tumor fingerprint for the sample of interest 608.

To illustrate, to determine the non-tumor nucleotide reads 610 for the class 0 dataset 606, the MRD detection system 106 can generate a plurality of nucleotide reads based on the sample of interest 608. In particular, the MRD detection system 106 can generate a plurality of paired-end reads based on the sample of interest 608. In one or more embodiments, the MRD detection system 106 generates the plurality of paired-end reads via whole genome sequencing.

Further, the MRD detection system 106 can identify or select the non-tumor nucleotide reads 610 from the plurality of paired-end reads generated from the sample of interest 608. For instance, the MRD detection system 106 can analyze the plurality of paired-end reads generated from the sample of interest 608 to identify which paired-end reads are non-tumor nucleotide reads. Indeed, the MRD detection system 106 can analyze the plurality of paired-end reads to identify paired-end reads at pseudo fingerprint genomic regions within the sample of interest. The MRD detection system 106 can include one or more of these paired-end reads as the non-tumor nucleotide reads 610 within the class 0 dataset 606 generated for sample of interest 608.

As further shown in FIG. 6, the MRD detection system 106 similarly generates class 0 datasets 612a-612n for the additional machine learning models 604a-604n using the samples 614a-614n. In particular, as indicated, the MRD detection system 106 generates a separate class 0 dataset for each of the additional machine learning models 604a-604n using a corresponding sample from the samples 614a-614n. In one or more embodiments, the samples 614a-614n include various samples from a panel of normals 616. Indeed, in some cases, the samples 614a-614n include samples obtained from non-cancer subjects. In some implementations, the samples 614a-614n include blood samples (e.g., plasma samples) taken from subjects associated with the panel of normals 616.

As FIG. 6 illustrates, the MRD detection system 106 generates the class 0 datasets 612a-612n by determining non-tumor nucleotide reads 618a-618n from the samples 614a-614n. Indeed, the MRD detection system 106 can generate, for each of the additional machine learning models 604a-604n, a class 0 dataset that includes non-tumor nucleotide reads as described above with reference to generating the class 0 dataset 606 for the first machine learning model 602. In particular, the MRD detection system 106 can generate a plurality of nucleotide reads (e.g., paired-end reads) from a given sample, identify which of those nucleotide reads are non-tumor nucleotide reads, and include one or more of those non-tumor nucleotide reads as the non-tumor nucleotide reads of the corresponding class 0 dataset.

Notably, just as the MRD detection system 106 associates the first machine learning model 602 with the sample of interest 608 by generating the class 0 dataset 606 for the first machine learning model 602 using the sample of interest 608, the MRD detection system 106 also associates each of the additional machine learning models 604a-604n with a particular sample from the samples 614a-614n by generating a class 0 dataset for each of the additional machine learning model using a particular sample. In other words, each machine learning model corresponds to a particular sample and is trained on data from that sample.

As should be further noted, the MRD detection system 106 can determine the non-tumor nucleotide reads 618a-618n to include within the class 0 datasets 612a-612n with respect to the tumor fingerprint for the sample of interest 608. Indeed, as indicated in FIG. 6, the non-tumor nucleotide reads 618a-618n are at pseudo fingerprint genomic regions within their respective samples, where the pseudo fingerprint genomic regions are determined with respect to the tumor fingerprint for the sample of interest 608. In other words, the non-tumor nucleotide reads 618a-618n do not overlap with the target genomic region associated with the tumor fingerprint for the sample of interest 608.

Additionally, in one or more embodiments, the MRD detection system 106 includes a nucleotide read within a class 0 dataset by including features extracted from the nucleotide read (e.g., as discussed above with reference to FIG. 4) within the class 0 dataset.

As shown in FIG. 6, the MRD detection system 106 trains each of the machine learning models using its corresponding class 0 dataset. For example, the MRD detection system 106 trains the first machine learning model 602 using the class 0 dataset 606 generated from the sample of interest 608. Additionally, the MRD detection system 106 trains the additional machine learning model 604a using the class 0 dataset 612a generated from the sample 614a. Further, the MRD detection system 106 trains the additional machine learning model 604n using the class 0 dataset 612n generated from the sample 614n.

As further illustrated, the MRD detection system 106 trains each of the machine learning models using a class 1 dataset 620 (e.g., the class 1 dataset 502 discussed above with reference to FIG. 5). Indeed, as previously mentioned, the MRD detection system 106 can use the class 1 dataset as a shared dataset to train all machine learning models that are to be implemented for MRD detection. Thus, as indicated by FIG. 6, the MRD detection system 106 can train each machine learning model using the class 1 dataset 620 and a corresponding class 0 dataset.

For instance, the MRD detection system 106 can use sample of interest training data having data from the class 0 dataset 606 and the class 1 dataset 620 to train the first machine learning model 602. Similarly, the MRD detection system 106 can use panel of normals training data having data from the class 0 datasets 612a-612n and the class 1 dataset 620 to train the additional machine learning models 604a-604n. In particular, the MRD detection system 106 can determine a set of panel of normals training data for an additional machine learning model to include data from the class 1 dataset 620 as well as data from the corresponding class 0 dataset.

In one or more embodiments, the MRD detection system 106 trains a machine learning model by providing a nucleotide read (e.g., features extracted from the nucleotide read) taken from either the class 1 dataset 502 or the corresponding class 0 dataset as input. The MRD detection system 106 further uses the machine learning model to generate a prediction as to whether the input nucleotide read is a tumor supporting read or a non-tumor supporting read. Indeed, the MRD detection system 106 can use the machine learning model to classify the input nucleotide read. The MRD detection system 106 can compare the predicted classification to a ground truth label for the input nucleotide read, which the MRD detection system 106 can determine from the identity of the dataset from which the input nucleotide was taken. In other words, the MRD detection system 106 can determine that nucleotide reads coming from the class 1 dataset 620 are tumor supporting nucleotide reads and nucleotide reads coming from the corresponding class 0 dataset are non-tumor nucleotide reads. Based on the comparison of the predicted classification to the ground truth label, the MRD detection system 106 can determine an error of the machine learning model and update its parameters via back propagation. Over several iterations, the MRD detection system 106 can reduce the error of the machine learning model and learn its parameters.

By training the machine learning models on corresponding class 0 datasets that include nucleotide reads determined with respect to the tumor fingerprint for a subject of interest, the MRD detection system 106 personalizes the training to the subject of interest. In particular, the MRD detection system 106 trains the machine learning models to recognize nucleotide patterns that do not correspond to the type of cancer for which the subject of interest has been diagnosed. Further, by training each of the machine learning models on a class 1 dataset and a corresponding class 0 dataset, the MRD detection system 106 trains the machine learning models to recognize both tumor supporting nucleotide patterns (e.g., nucleotide patterns that indicate the presence of ctDNA) and non-tumor nucleotide patterns.

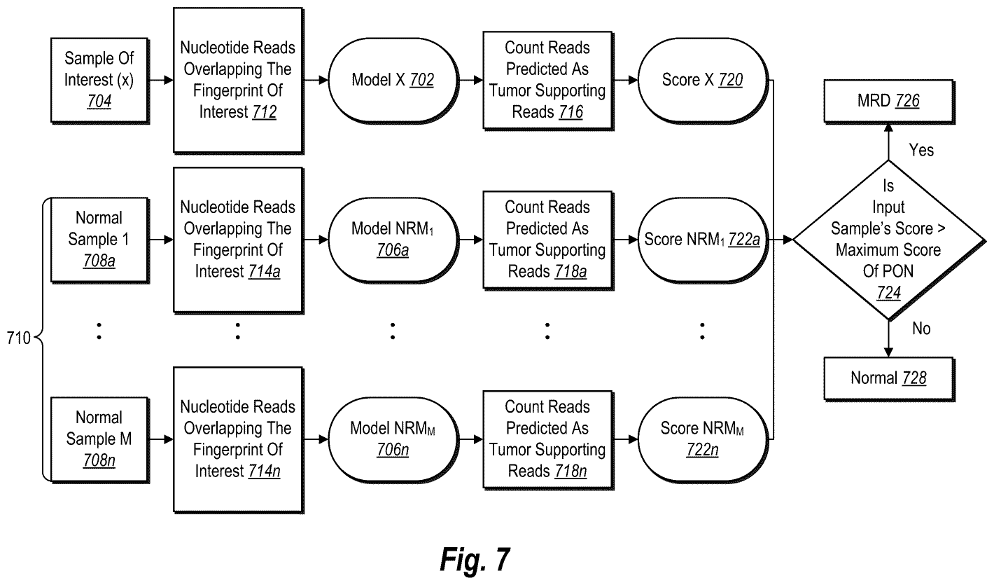

In one or more embodiments, the MRD detection system 106 implements the trained collection of machine learning models to process various samples in testing the subject of interest for MRD during inference. FIG. 7 illustrates the MRD detection system 106 using a collection of machine learning models to process various samples to test a subject of interest for MRD in accordance with one or more embodiments.

As shown in FIG. 7, the MRD detection system 106 uses a first machine learning model 702 to process a sample of interest 704 from the subject of interest. Further, the MRD detection system 106 uses additional machine learning models 706a-706n to process samples 708a-708n, respectively, from a panel of normals 710. Notably, in one or more embodiments, the sample of interest 704 corresponds to the sample of interest 608 discussed with reference to FIG. 6. Further, the samples 708a-708n correspond to the samples 614a-614n discussed with reference to FIG. 6. Indeed, in one or more embodiments, the MRD detection system 106 uses each machine learning model to test the particular sample upon which the machine learning model was trained. Said differently, the MRD detection system 106 can train each machine learning model on nucleotide reads from the particular sample that will be tested during inference.

As indicated in FIG. 7, the MRD detection system 106 uses a machine learning model to process the corresponding sample by using the machine learning model to process nucleotide reads (e.g., paired-end reads) generated from the corresponding sample. For instance, as shown, the MRD detection system 106 uses the first machine learning model 702 to process nucleotide reads 712 generated from the sample of interest 704 (i.e., sample of interest nucleotide reads). Further, the MRD detection system 106 uses the additional machine learning models 706a-706n to process nucleotide reads 714a-714n generated from the samples 708a-708n, respectively (i.e., panel of normals nucleotide reads). As indicated, the nucleotide reads 712, 714a-714n include nucleotide reads that overlap the tumor fingerprint associated with the sample of interest 704. In other words, the nucleotide reads 712, 714a-714n include nucleotide reads that overlap the target genomic region associated with the tumor fingerprint. As previously discussed, the MRD detection system 106 can use a machine learning model to process a nucleotide read by using the machine learning model to process features extracted from the nucleotide read.

To illustrate, in determining the nucleotide reads to provide to a machine learning model, the MRD detection system 106 can generate a plurality of nucleotide reads (e.g., paired end reads) from the corresponding sample. The MRD detection system 106 further determines which of those nucleotide reads overlap with the target genomic region associated with the tumor fingerprint determined for the subject of interest. For instance, the MRD detection system 106 can identify paired-end reads having a first read and a second read that both overlap the target genomic region. In some cases, the MRD detection system 106 identifies overlapping nucleotide reads by aligning the nucleotide reads generated from the sample with a reference genome as discussed above with reference to FIG. 3.

As indicated by FIG. 6, the MRD detection system 106 uses each of the machine learning models to classify each of the nucleotide reads processed by that machine learning model as a tumor supporting nucleotide read or a non-tumor nucleotide read. As further shown, the MRD detection system 106 performs an act of counting the number of nucleotide reads classified by each machine learning model as tumor supporting nucleotide reads. Indeed, the MRD detection system 106 performs an act 716 of counting the number of nucleotide reads classified as tumor supporting nucleotide reads by the first machine learning model 702. Similarly, the MRD detection system 106 performs acts 718a-718n of counting the number of nucleotide reads classified as tumor supporting nucleotide reads by the additional machine learning models 706a-706n, respectively. In one or more embodiments, the MRD detection system 106 provides the same number of nucleotide reads to each of the machine learning models to ensure consistency among the number of classifications produced by each machine learning model.

As further illustrated in FIG. 6, based on counting the number of nucleotide reads classified as tumor supporting nucleotide reads by each machine learning model, the MRD detection system 106 determines a score for each sample processed by the machine learning models. Indeed, as shown, the MRD detection system 106 determines a score 720 for the sample of interest 704 (i.e., a sample of interest score) that reflects the number of nucleotide reads generated from the sample of interest 704 that are classified as tumor supporting nucleotide reads. Similarly, the MRD detection system 106 determines scores 722a-722n for the samples 708a-708n (i.e., panel of normals scores) that respectively reflect the number of nucleotide reads generated from the samples 708a-708n that are classified as tumor supporting nucleotide reads.

As further shown, the MRD detection system 106 performs an act 724 of comparing the scores determined from the machine learning model outputs. In particular, the MRD detection system 106 determines the highest score determined from the outputs of the additional machine learning models 706a-706n (i.e., a maximum panel of normals score). The MRD detection system 106 further compares the score 720 determined from the outputs of the first machine learning model 702 (i.e., the sample of interest score) to the highest score from the additional machine learning models 706a-706n. Based on the comparison, the MRD detection system 106 determines whether the sample of interest 704 has MRD related to the type of cancer infecting the subject of interest. In particular, based on determining that the score 720 determined for the first machine learning model 702 is higher than the scores determined from the additional machine learning models 706a-706n, the MRD detection system 106 can provide a prediction 726 indicating that MRD has been detected. In some cases, the MRD detection system 106 provides the prediction 726 based on determining that the score 720 is more than a threshold greater in value than the highest score for the additional machine learning models 706a-706n. Conversely, based on determining that the score determined for the first machine learning model is lower than the scores determined from the additional machine learning models 706a-706n (or lower or equal to the threshold), the MRD detection system 106 can provide a prediction 728 indicating that MRD has not been detected. In other words, the MRD detection system 106 can predict that the sample of interest 704 is normal (does not have MRD).

Thus, the MRD detection system 106 implements a new, unconventional approach to detecting MRD within subjects that have been diagnosed with cancer. In particular, the MRD detection system 106 implements an unconventional combination of steps that involves using a sophisticated combination of machine learning models that have been trained on a unique combination of training data to process various samples from various subjects to determine whether a sample of interest has MRD. Indeed, where many existing systems focus on the sequencing determined for a sample of interest to detect the presence of MRD-supporting circulating tumor DNA (ctDNA), the MRD detection system implements a plurality of machine learning models to compare the sample of interest to various other samples.

By implementing the machine learning models as described above, the MRD detection system 106 more flexibly accommodates noisy nucleotide reads. Indeed, by using the collection of machine learning models to generate scores for a sample of interest and various samples from a panel of normals and then comparing those scores to determine whether MRD has been detected, the MRD detection system 106 accommodates the presence of noisy nucleotide reads that may falsely indicate the presence of ctDNA within their respective samples. In particular, by using additional machine learning models to process samples from a panel of normals, the MRD detection system 106 can establish a baseline for the presence of noisy nucleotide reads within non-tumor samples. Thus, the MRD detection system 106 by comparing the sample of interest to the samples from the panel of normals, the MRD detection system 106 can flexibly determine whether the sample of interest indicates the presence of ctDNA beyond what is considered normal per the baseline. By accommodating noisy nucleotide reads in this way, the MRD detection system 106 can provide more accurate MRD detection results.

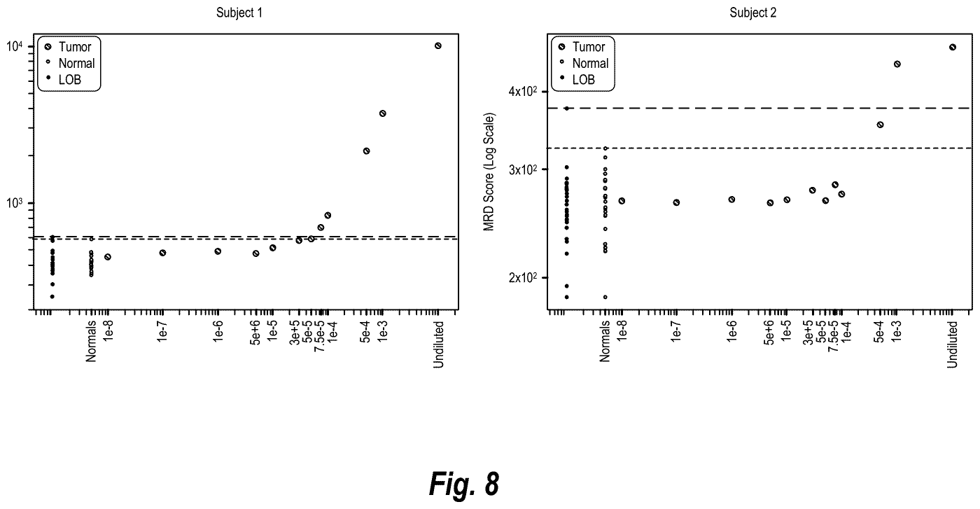

FIG. 8 illustrates graphs reflecting experimental results regarding the effectiveness of the MRD detection system 106 in accordance with one or more embodiments. In particular, each graph illustrated in FIG. 8 corresponds to a particular subject that has been diagnosed with cancer. Additionally, each graph provides scores (labeled here as “MRD score”) that are determined for variations of a sample (e.g., plasma sample) from the corresponding subject. As indicated by the x-axis of each graph, the variations of the sample include an undiluted variation of the sample (far right) and variations resulting from different levels of dilution (with positions farther to the left indicating more dilution). In particular, the diluted variations correspond to variations in which the tumor burden has been diluted.

Each graph compares the scores determined for the sample of the corresponding subject to scores determined for samples from a panel of normals. The horizontal lines (e.g., the bottom lines) in the graphs each indicate the maximum score determined for corresponding panel or normals samples. In other words, the horizontal lines indicate the score that would be needed for a sample from the corresponding subject to indicate that the sample has MRD in certain embodiments. Indeed, in some cases, the MRD detection system 106 determines that each of the sample variations above the horizontal lines are MRD positive and each of the sample variations below the horizontal lines are MRD negative (i.e., normal).

As shown by the graphs of FIG. 8, the MRD detection system 106 is produces scores for the undiluted sample variations of each subject that are well above the corresponding horizontal line, providing a clear indication that these sample variations are MRD positive. Additionally, as the tumor burden with the samples is reduced, the scores lower, showing that the MRD detection system 106 is responsive to the level of tumor burden within a sample.

In addition or in the alternative to improving the accuracy and sensitivity of MRD detection, in some embodiments, the present disclosure further includes one or more embodiments in which the MRD system 106 performs or facilitates methods of treating cancer or other forms of MRD in a subject. Such methods may comprise obtaining a biological sample (e.g., a FFPE tissue sample) of a cancer from the subject before the subject is administered a first anti-cancer therapy; detecting one or more variants (e.g., a subset of variants) in the biological sample to generate a tumor profile for the subject before the subject is administered the first anti-cancer therapy; administering the first anti-cancer therapy to the subject; obtaining a liquid biopsy from the subject after the subject was administered the first anti-cancer therapy; detecting the one or more variants in the liquid biopsy, wherein the cancer has recurred and wherein the one or more variants detected in the liquid biopsy are present in the tumor profile; and administering a second anti-cancer therapy to the subject after recurrence of the cancer.

In some embodiments, “treating” or “treatment” of a disease, disorder, or condition includes, at least partially, (1) preventing the disease, disorder, or condition, e.g., causing the clinical symptoms of the disease, disorder, or condition not to develop in a mammal that is exposed to or predisposed to the disease, disorder, or condition but does not yet experience or display symptoms of the disease, disorder, or condition; (2) inhibiting the disease, disorder, or condition, e.g., arresting or reducing the development of the disease, disorder, or condition or its clinical symptoms; or (3) relieving the disease, disorder, or condition, e.g., causing regression of the disease, disorder, or condition or its clinical symptoms. The treating or treatment of a disease or disorder may include treating or the treatment of cancer.

The term “treating cancer” refers to administration to a mammal afflicted with a cancerous condition and refers to an effect that alleviates the cancerous condition by killing the cancerous cells, but also to an effect that results in the inhibition of growth and/or metastasis of the cancer.

The anti-cancer therapy (e.g., first anti-cancer therapy or second anti-cancer therapy) can include any well-known therapies to treat cancer, including, but not limited to, surgical removal of the cancer, administration of chemotherapy, administration of radiation, administration of antibody therapies, and administration of anti-cancer drugs. In some embodiments, the second anti-cancer therapy is different than the first anti-cancer therapy.

The term “chemotherapy” refers to the treatment of cancer or a disease or disorder caused by a virus, bacterium, other microorganism, or an inappropriate immune response using specific chemical agents, drugs, or radioactive agents that are selectively toxic and destructive to malignant cells and tissues, viruses, bacteria, or other microorganisms. Chemotherapeutic agents or drugs, such as an anti-folate (e.g., Methotrexate) or any other agent or drug useful in treating cancer, an inflammatory disease, or an autoimmune disease are preferred. Suitable chemotherapeutic agents and drugs include, but are not limited to, actinomycin D, adriamycin, altretamine, azathioprine, bleomycin, busulphan, capecitabine, carboplatin, carmustine, chlorambucil, cisplatin, cladribine, crisantaspase, cyclophosphamide, cytarabine, dacarbazine, daunorubicin, doxorubicin, epirubicin, etoposide, fludarabine, fluorouracil, gemcitabine, hydroxyurea, idarubicin, ifosfamide, irinotecan, liposomal doxorubicin, lomustine, melphalan, mercaptopurine, Methotrexate, mitomycin, mitozantrone, oxaliplatin, paclitaxel, pentostatin, procarbazine, raltitrexed, steroids, streptozocin, taxol, taxotere, temozolomide, thioguanine, thiotepa, tomudex, topotecan, treosulfan, uft (uracil-tegufur), vinblastine, vincristine, vindesine, and vinorelbine.

The present disclosure further includes one or more embodiments in which the MRD system 106 performs or facilitates methods for administering an anti-cancer therapy to a subject with cancer. Such methods may comprise obtaining a liquid biopsy from a subject after a predetermined period of time and after the subject was administered a first anti-cancer therapy (e.g., after the subject's cancer has gone into remission); detecting one or more variants (e.g., a subset of variants) in the liquid biopsy, wherein recurrence of the cancer has occurred and wherein the one or more variants detected in the liquid biopsy are present in a tumor profile created from information obtained from the subject prior to treatment with the first anti-cancer therapy; and administering a second anti-cancer therapy to the subject after recurrence of the cancer is detected.

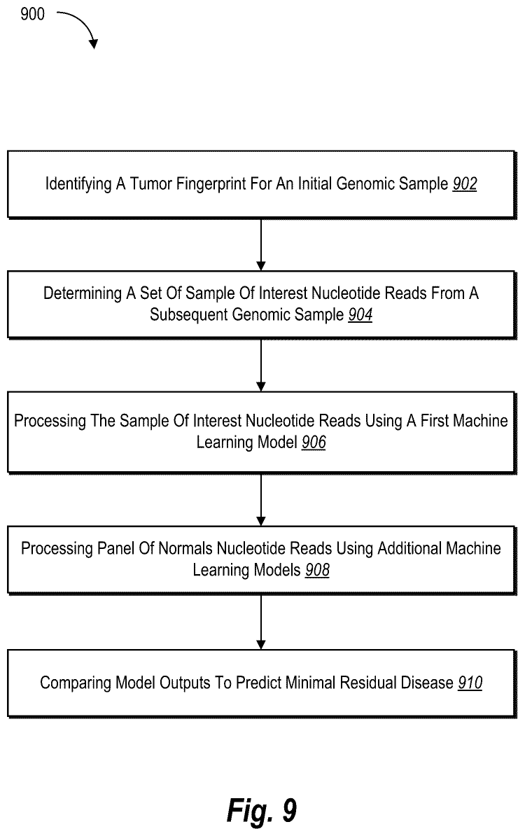

FIGS. 1-8, the corresponding text and the examples provide a number of different methods, systems, devices, and non-transitory computer-readable media of the MRD detection system 106. In addition to the foregoing, one or more embodiments can also be described in terms of flowcharts comprising acts for accomplishing particular results, as shown in FIG. 9. FIG. 9 may be performed with more or fewer acts. Further, the acts may be performed in different orders. Additionally, the acts described herein may be repeated or performed in parallel with one another or in parallel with different instances of the same or similar acts.

FIG. 9 illustrates a flowchart of a series of acts 900 for detecting minimal residual disease within a sample of interest in accordance with one or more embodiments. While FIG. 9 illustrates acts according to one embodiment, alternative embodiments may omit, add to, reorder, and/or modify any of the acts shown in FIG. 9. In some implementations, the acts of FIG. 9 are performed as part of a computer-implemented method. In some instances, a non-transitory computer-readable medium stores instructions thereon that, when executed by at least one processor, cause a computing device to perform the acts of FIG. 9. In some implementations, a system performs the acts of FIG. 9. For example, in one or more cases, a system includes at least one processor and a non-transitory computer-readable medium storing instructions that, when executed by the at least one processor, cause the system to perform the acts of FIG. 9.

As shown in FIG. 9, the series of acts 900 includes an act 902 of identifying a tumor fingerprint for an initial genomic sample; an act 904 of determining a set of sample of interest nucleotide reads from a subsequent genomic sample; and act 906 of processing the sample of interest nucleotide reads using a first machine learning model; and act 908 of processing panel of normals nucleotide reads using additional machine learning models; and an act 910 of comparing model outputs to predict minimal residual disease. For example, the series of acts 900 can include acts to perform any of the operations described in the following clauses:

CLAUSE 1. A computer-implemented method comprising:
identifying, for an initial genomic sample of a subject infected with a type of cancer, a tumor fingerprint comprising variants at a target genomic region;
determining, for a sample of interest corresponding to a subsequent genomic sample of the subject, a set of sample of interest nucleotide reads associated with the target genomic region;
processing, using a first machine learning model that was trained with sample of interest training data, extracted features from the set of sample of interest nucleotide reads to generate a sample of interest score;
processing, using one or more additional machine learning models that were trained with panel of normals training data, extracted features from panel of normals nucleotide reads associated with the target genomic region to generate one or more panel of normals scores; and
comparing the sample of interest score to the one or more panel of normals scores to predict whether the sample of interest has minimal residual disease related to the type of cancer.

CLAUSE 2. The computer-implemented method of clause 1, wherein the sample of interest training data used to train the first machine learning model comprises:
a class 1 dataset corresponding to tumor supporting nucleotide reads; and
a first class 0 dataset corresponding to non-tumor nucleotide reads from pseudo fingerprint genomic regions within the sample of interest.

CLAUSE 3. The computer-implemented method of clause 2, wherein the pseudo fingerprint genomic regions within the sample of interest comprise regions that do not overlap the target genomic region associated with the tumor fingerprint.

CLAUSE 4. The computer-implemented method of clause 3, wherein the panel of normals training data used to train the one or more additional machine learning models comprises: the class 1 dataset corresponding to the tumor supporting nucleotide reads; and
one or more additional class 0 datasets corresponding to one or more samples within a panel of normals, where each of the one or more additional machine learning models corresponds to a specific sample from the one or more samples within the panel of normals.

CLAUSE 5. The computer-implemented method of clause 2, wherein the tumor supporting nucleotide reads of the class 1 dataset includes a plurality of paired-end reads corresponding to a plurality of tumor samples, wherein each paired-end read corresponds to a tumor sample and comprises a first read and a second read that overlap with a genomic region of a fingerprint for the tumor sample and support a tumor allele associated with tumor sample.

CLAUSE 6. The computer-implemented method of clause 1, wherein the initial genomic sample comprises a sample from a tumor of the subject and the subsequent genomic sample comprises a plasma sample comprising cell-free deoxyribonucleic acid (cfDNA).

CLAUSE 7. The computer-implemented method of clause 1, further comprising instructions that, when executed by the at least one processor, cause the system to extract features from the set of sample of interest nucleotide reads associated with the target genomic region.

CLAUSE 8. The computer-implemented method of clause 7, wherein extracting the features from the set of sample of interest nucleotide reads associated with the target genomic region comprises extracting the features from a plurality of paired-end reads, each paired-end read comprising a first read and a second read that overlap with the target genomic region of the tumor fingerprint.

CLAUSE 9. The computer-implemented method of clause 8, wherein the extracted features comprise at least one of:
individual read features for the first read of each of the plurality of paired-end reads;
individual read features for the second read of each of the plurality of paired-end reads;
or
combination read features corresponding to properties associated with the combination of the first read and the second read.

The methods described herein can be used in conjunction with a variety of nucleic acid sequencing techniques. Particularly applicable techniques are those wherein nucleic acids are attached at fixed locations in an array such that their relative positions do not change and wherein the array is repeatedly imaged. Implementations in which images are obtained in different color channels, for example, coinciding with different labels used to distinguish one nucleobase type from another are particularly applicable. In some implementations, the process to determine the nucleotide sequence of a target nucleic acid (i.e., a nucleic-acid polymer) can be an automated process. Preferred implementations include sequencing-by-synthesis (SBS) techniques.

SBS techniques generally involve the enzymatic extension of a nascent nucleic acid strand through the iterative addition of nucleotides against a template strand. In traditional methods of SBS, a single nucleotide monomer may be provided to a target nucleotide in the presence of a polymerase in each delivery. However, in the methods described herein, more than one type of nucleotide monomer can be provided to a target nucleic acid in the presence of a polymerase in a delivery.

SBS can utilize nucleotide monomers that have a terminator moiety or those that lack any terminator moieties. Methods utilizing nucleotide monomers lacking terminators include, for example, pyrosequencing and sequencing using γ-phosphate-labeled nucleotides, as set forth in further detail below. In methods using nucleotide monomers lacking terminators, the number of nucleotides added in each cycle is generally variable and dependent upon the template sequence and the mode of nucleotide delivery. For SBS techniques that utilize nucleotide monomers having a terminator moiety, the terminator can be effectively irreversible under the sequencing conditions used as is the case for traditional Sanger sequencing which utilizes dideoxynucleotides, or the terminator can be reversible as is the case for sequencing methods developed by Solexa (now Illumina, Inc.).

SBS techniques can utilize nucleotide monomers that have a label moiety or those that lack a label moiety. Accordingly, incorporation events can be detected based on a characteristic of the label, such as fluorescence of the label; a characteristic of the nucleotide monomer such as molecular weight or charge; a byproduct of incorporation of the nucleotide, such as the release of pyrophosphate; or the like. In implementations, where two or more different nucleotides are present in a sequencing reagent, the different nucleotides can be distinguishable from each other, or alternatively, the two or more different labels can be indistinguishable under the detection techniques being used. For example, the different nucleotides present in a sequencing reagent can have different labels and they can be distinguished using appropriate optics as exemplified by the sequencing methods developed by Solexa (now Illumina, Inc.).

Preferred implementations include pyrosequencing techniques. Pyrosequencing detects the release of inorganic pyrophosphate (PPi) as particular nucleotides are incorporated into the nascent strand (Ronaghi, M., Karamohamed, S., Pettersson, B., Uhlen, M. and Nyren, P. (1996) “Real-time DNA sequencing using detection of pyrophosphate release.” Analytical Biochemistry 242 (1), 84-9; Ronaghi, M. (2001) “Pyrosequencing sheds light on DNA sequencing.” Genome Res. 11 (1), 3-11; Ronaghi, M., Uhlen, M. and Nyren, P. (1998) “A sequencing method based on real-time pyrophosphate.” Science 281 (5375), 363; U.S. Pat. Nos. 6,210,891; 6,258,568 and 6,274,320, the disclosures of which are incorporated herein by reference in their entireties). In pyrosequencing, released PPi can be detected by being immediately converted to adenosine triphosphate (ATP) by ATP sulfurylase, and the level of ATP generated is detected via luciferase-produced photons. The nucleic acids to be sequenced can be attached to features in an array and the array can be imaged to capture the chemiluminescent signals that are produced due to the incorporation of nucleotides at the features of the array. An image can be obtained after the array is treated with a particular nucleotide type (e.g., A, T, C, or G). Images obtained after the addition of each nucleotide type will differ with regard to which features in the array are detected. These differences in the image reflect the different sequence content of the features on the array. However, the relative locations of each feature will remain unchanged in the images. The images can be stored, processed, and analyzed using the methods set forth herein. For example, images obtained after treatment of the array with each different nucleotide type can be handled in the same way as exemplified herein for images obtained from different detection channels for reversible terminator-based sequencing methods.

In another exemplary type of SBS, cycle sequencing is accomplished by stepwise addition of reversible terminator nucleotides containing, for example, a cleavable or photobleachable dye label as described, for example, in WO 04/018497 and U.S. Pat. No. 7,057,026, the disclosures of which are incorporated herein by reference. This approach is being commercialized by Solexa (now Illumina Inc.) and is also described in WO 91/06678 and WO 07/123,744, each of which is incorporated herein by reference. The availability of fluorescently-labeled terminators in which both the termination can be reversed, and the fluorescent label cleaved facilitates efficient cyclic reversible termination (CRT) sequencing. Polymerases can also be co-engineered to efficiently incorporate and extend from these modified nucleotides.

Preferably in reversible terminator-based sequencing implementations, the labels do not substantially inhibit extension under SBS reaction conditions. However, the detection labels can be removable, for example, by cleavage or degradation. Images can be captured following the incorporation of labels into arrayed nucleic acid features. In particular implementations, each cycle involves simultaneous delivery of four different nucleotide types to the array and each nucleotide type has a spectrally distinct label. Four images can then be obtained, each using a detection channel that is selective for one of the four different labels. Alternatively, different nucleotide types can be added sequentially and an image of the array can be obtained between each addition step. In such implementations, each image will show nucleic acid features that have incorporated nucleotides of a particular type. Different features are present or absent in the different images due to the different sequence content of each feature. However, the relative position of the features will remain unchanged in the images. Images obtained from such reversible terminator-SBS methods can be stored, processed, and analyzed as set forth herein. Following the image capture step, labels can be removed, and reversible terminator moieties can be removed for subsequent cycles of nucleotide addition and detection. Removal of the labels after they have been detected in a particular cycle and prior to a subsequent cycle can provide the advantage of reducing background signal and crosstalk between cycles. Examples of useful labels and removal methods are set forth below.

In particular implementations, some or all of the nucleotide monomers can include reversible terminators. In such implementations, reversible terminators/cleavable fluors can include fluor linked to the ribose moiety via a 3′ ester linkage (Metzker, Genome Res. 15:1767-1776 (2005), which is incorporated herein by reference). Other approaches have separated the terminator chemistry from the cleavage of the fluorescence label (Ruparel et al., Proc Natl Acad Sci USA 102:5932-7 (2005), which is incorporated herein by reference in its entirety). Ruparel et al described the development of reversible terminators that used a small 3′ allyl group to block extension, but could easily be deblocked by a short treatment with a palladium catalyst. The fluorophore was attached to the base via a photocleavable linker that could easily be cleaved by a 30-second exposure to long-wavelength UV light. Thus, either disulfide reduction or photocleavage can be used as a cleavable linker. Another approach to reversible termination is the use of natural termination that ensues after the placement of a bulky dye on a dNTP. The presence of a charged bulky dye on the dNTP can act as an effective terminator through steric and/or electrostatic hindrance. The presence of one incorporation event prevents further incorporations unless the dye is removed. Cleavage of the dye removes the fluor and effectively reverses the termination. Examples of modified nucleotides are also described in U.S. Pat. Nos. 7,427,673, and 7,057,026, the disclosures of which are incorporated herein by reference in their entireties.

Additional exemplary SBS systems and methods which can be utilized with the methods and systems described herein are described in U.S. Patent Application Publication No. 2007/0166705, U.S. Patent Application Publication No. 2006/0188901, U.S. Pat. No. 7,057,026, U.S. Patent Application Publication No. 2006/0240439, U.S. Patent Application Publication No. 2006/0281109, PCT Publication No. WO 05/065814, U.S. Patent Application Publication No. 2005/0100800, PCT Publication No. WO 06/064199, PCT Publication No. WO 07/010,251, U.S. Patent Application Publication No. 2012/0270305 and U.S. Patent Application Publication No. 2013/0260372, the disclosures of which are incorporated herein by reference in their entireties.

Some implementations can utilize the detection of four different nucleotides using fewer than four different labels. For example, SBS can be performed utilizing methods and systems described in the incorporated materials of U.S. Patent Application Publication No. 2013/0079232. As a first example, a pair of nucleotide types can be detected at the same wavelength, but distinguished based on a difference in intensity for one member of the pair compared to the other, or based on a change to one member of the pair (e.g. via chemical modification, photochemical modification or physical modification) that causes an apparent signal to appear or disappear compared to the signal detected for the other member of the pair. As a second example, three of four different nucleotide types can be detected under particular conditions while a fourth nucleotide type lacks a label that is detectable under those conditions, or is minimally detected under those conditions (e.g., minimal detection due to background fluorescence, etc.). Incorporation of the first three nucleotide types into a nucleic acid can be determined based on the presence of their respective signals and incorporation of the fourth nucleotide type into the nucleic acid can be determined based on the absence or minimal detection of any signal. As a third example, one nucleotide type can include label(s) that are detected in two different channels, whereas other nucleotide types are detected in no more than one of the channels. The aforementioned three exemplary configurations are not considered mutually exclusive and can be used in various combinations. An exemplary implementation that combines all three examples, is a fluorescent-based SBS method that uses a first nucleotide type that is detected in a first channel (e.g. dATP having a label that is detected in the first channel when excited by a first excitation wavelength), a second nucleotide type that is detected in a second channel (e.g. dCTP having a label that is detected in the second channel when excited by a second excitation wavelength), a third nucleotide type that is detected in both the first and the second channel (e.g. dTTP having at least one label that is detected in both channels when excited by the first and/or second excitation wavelength) and a fourth nucleotide type that lacks a label that is not, or minimally, detected in either channel (e.g. dGTP having no label).

Further, as described in the incorporated materials of U.S. Patent Application Publication No. 2013/0079232, sequencing data can be obtained using a single channel. In such so-called one-dye sequencing approaches, the first nucleotide type is labeled but the label is removed after the first image is generated, and the second nucleotide type is labeled only after a first image is generated. The third nucleotide type retains its label in both the first and second images, and the fourth nucleotide type remains unlabeled in both images.

Some implementations can utilize sequencing by ligation techniques. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. As with other SBS methods, images can be obtained following treatment of an array of nucleic acid features with the labeled sequencing reagents. Each image will show nucleic acid features that have incorporated labels of a particular type. Different features are present or absent in the different images due to the different sequence content of each feature, but the relative position of the features will remain unchanged in the images. Images obtained from ligation-based sequencing methods can be stored, processed, and analyzed as set forth herein. Exemplary SBS systems and methods which can be utilized with the methods and systems described herein are described in U.S. Pat. Nos. 6,969,488, 6,172,218, and 6,306,597, the disclosures of which are incorporated herein by reference in their entireties.

Some implementations can utilize nanopore sequencing (Deamer, D. W. & Akeson, M. “Nanopores and nucleic acids: prospects for ultrarapid sequencing.” Trends Biotechnol. 18, 147-151 (2000); Deamer, D. and D. Branton, “Characterization of nucleic acids by nanopore analysis”. Acc. Chem. Res. 35:817-825 (2002); Li, J., M. Gershow, D. Stein, E. Brandin, and J. A. Golovchenko, “DNA molecules and configurations in a solid-state nanopore microscope” Nat. Mater. 2:611-615 (2003), the disclosures of which are incorporated herein by reference in their entireties). In such implementations, the target nucleic acid passes through a nanopore. The nanopore can be a synthetic pore or biological membrane protein, such as α-hemolysin. As the target nucleic acid passes through the nanopore, each base-pair can be identified by measuring fluctuations in the electrical conductance of the pore. (U.S. Pat. No. 7,001,792; Soni, G. V. & Meller, “A. Progress toward ultrafast DNA sequencing using solid-state nanopores.” Clin. Chem. 53, 1996-2001 (2007); Healy, K. “Nanopore-based single-molecule DNA analysis.” Nanomed. 2, 459-481 (2007); Cockroft, S. L., Chu, J., Amorin, M. & Ghadiri, M. R. “A single-molecule nanopore device detects DNA polymerase activity with single-nucleotide resolution.” J. Am. Chem. Soc. 130, 818-820 (2008), the disclosures of which are incorporated herein by reference in their entireties). Data obtained from nanopore sequencing can be stored, processed, and analyzed as set forth herein. In particular, the data can be treated as an image in accordance with the exemplary treatment of optical images and other images that is set forth herein.

Some implementations can utilize methods involving the real-time monitoring of DNA polymerase activity. Nucleotide incorporations can be detected through fluorescence resonance energy transfer (FRET) interactions between a fluorophore-bearing polymerase and γ-phosphate-labeled nucleotides as described, for example, in U.S. Pat. Nos. 7,329,492 and 7,211,414 (each of which is incorporated herein by reference) or nucleotide incorporations can be detected with zero-mode waveguides as described, for example, in U.S. Pat. No. 7,315,019 (which is incorporated herein by reference) and using fluorescent nucleotide analogs and engineered polymerases as described, for example, in U.S. Pat. No. 7,405,281 and U.S. Patent Application Publication No. 2008/0108082 (each of which is incorporated herein by reference). The illumination can be restricted to a zeptoliter-scale volume around a surface-tethered polymerase such that incorporation of fluorescently labeled nucleotides can be observed with low background (Levene, M. J. et al. “Zero-mode waveguides for single-molecule analysis at high concentrations.” Science 299, 682-686 (2003); Lundquist, P. M. et al. “Parallel confocal detection of single molecules in real time.” Opt. Lett. 33, 1026-1028 (2008); Korlach, J. et al. “Selective aluminum passivation for targeted immobilization of single DNA polymerase molecules in zero-mode waveguide nano structures.” Proc. Natl. Acad. Sci. USA 105, 1176-1181 (2008), the disclosures of which are incorporated herein by reference in their entireties). Images obtained from such methods can be stored, processed, and analyzed as set forth herein.

Some SBS implementations include the detection of a proton released upon incorporation of a nucleotide into an extension product. For example, sequencing based on detection of released protons can use an electrical detector and associated techniques that are commercially available from Ion Torrent (Guilford, CT, a Life Technologies subsidiary) or sequencing methods and systems described in US 2009/0026082 A1; US 2009/0127589 A1; US 2010/0137143 A1; or US 2010/0282617 A1, each of which is incorporated herein by reference. Methods set forth herein for amplifying target nucleic acids using kinetic exclusion can be readily applied to substrates used for detecting protons. More specifically, methods set forth herein can be used to produce clonal populations of amplicons that are used to detect protons.

The above SBS methods can be advantageously carried out in multiplex formats such that multiple different target nucleic acids are manipulated simultaneously. In particular implementations, different target nucleic acids can be treated in a common reaction vessel or on a surface of a particular substrate. This allows convenient delivery of sequencing reagents, removal of unreacted reagents, and detection of incorporation events in a multiplex manner. In implementations using surface-bound target nucleic acids, the target nucleic acids can be in an array format. In an array format, the target nucleic acids can be typically bound to a surface in a spatially distinguishable manner. The target nucleic acids can be bound by direct covalent attachment, attachment to a bead or other particle, or binding to a polymerase or other molecule that is attached to the surface. The array can include a single copy of a target nucleic acid at each site (also referred to as a feature) or multiple copies having the same sequence can be present at each site or feature. Multiple copies can be produced by amplification methods such as bridge amplification or emulsion PCR as described in further detail below.

The methods set forth herein can use arrays having features at any of a variety of densities including, for example, at least about 10 features/cm2, 100 features/cm2, 500 features/cm2, 1,000 features/cm2, 5,000 features/cm2, 10,000 features/cm2, 50,000 features/cm2, 100,000 features/cm2, 1,000,000 features/cm2, 5,000,000 features/cm2, or higher.

An advantage of the methods set forth herein is that they provide for rapid and efficient detection of a plurality of target nucleic acid in parallel. Accordingly, the present disclosure provides integrated systems capable of preparing and detecting nucleic acids using techniques known in the art such as those exemplified above. Thus, an integrated system of the present disclosure can include fluidic components capable of delivering amplification reagents and/or sequencing reagents to one or more immobilized DNA fragments, the system comprising components such as pumps, valves, reservoirs, fluidic lines, and the like. A flow cell can be configured and/or used in an integrated system for the detection of target nucleic acids. Exemplary flow cells are described, for example, in US 2010/0111768 A1 and U.S. Ser. No. 13/273,666, each of which is incorporated herein by reference. As exemplified for flow cells, one or more of the fluidic components of an integrated system can be used for an amplification method and for a detection method. Taking a nucleic acid sequencing implementation as an example, one or more of the fluidic components of an integrated system can be used for an amplification method set forth herein and for the delivery of sequencing reagents in a sequencing method such as those exemplified above. Alternatively, an integrated system can include separate fluidic systems to carry out amplification methods and to carry out detection methods. Examples of integrated sequencing systems that are capable of creating amplified nucleic acids and also determining the sequence of the nucleic acids include, without limitation, the MiSeq™ platform (Illumina, Inc., San Diego, CA) and devices described in U.S. Ser. No. 13/273,666, which is incorporated herein by reference.

The sequencing system described above sequences nucleic-acid polymers present in samples received by a sequencing device. As defined herein, a “sample” (and its derivatives) is used in its broadest sense and includes any specimen, culture, and the like that is suspected of including a target. In some implementations, the sample comprises DNA, RNA, PNA, LNA, chimeric or hybrid forms of nucleic acids. The sample can include any biological, clinical, surgical, agricultural, atmospheric, or aquatic-based specimen containing one or more nucleic acids. The term also includes any isolated nucleic acid sample such a genomic DNA, fresh-frozen, or formalin-fixed paraffin-embedded nucleic acid specimen. It is also envisioned that the sample can be from a single individual, a collection of nucleic acid samples from genetically related members, nucleic acid samples from genetically unrelated members, nucleic acid samples (matched) from a single individual such as a tumor sample, and normal tissue sample, or sample from a single source that contains two distinct forms of genetic material such as maternal and fetal DNA obtained from a maternal subject, or the presence of contaminating bacterial DNA in a sample that contains plant or animal DNA. In some implementations, the source of nucleic acid material can include nucleic acids obtained from a newborn, for example as typically used for newborn screening.

The nucleic acid sample can include high molecular weight material such as genomic DNA (gDNA). The sample can include low molecular weight material such as nucleic acid molecules obtained from FFPE or archived DNA samples. In another implementation, low molecular weight material includes enzymatically or mechanically fragmented DNA. The sample can include cell-free circulating DNA. In some implementations, the sample can include nucleic acid molecules obtained from biopsies, tumors, scrapings, swabs, blood, mucus, urine, plasma, semen, hair, laser capture micro-dissections, surgical resections, and other clinical or laboratory obtained samples. In some implementations, the sample can be an epidemiological, agricultural, forensic, or pathogenic sample. In some implementations, the sample can include nucleic acid molecules obtained from an animal such as a human or mammalian source. In another implementation, the sample can include nucleic acid molecules obtained from a non-mammalian source such as a plant, bacteria, virus, or fungus. In some implementations, the source of the nucleic acid molecules may be an archived or extinct sample or species.

Further, the methods and compositions disclosed herein may be useful to amplify a nucleic acid sample having low-quality nucleic acid molecules, such as degraded and/or fragmented genomic DNA from a forensic sample. In one implementation, forensic samples can include nucleic acids obtained from a crime scene, nucleic acids obtained from a missing persons DNA database, nucleic acids obtained from a laboratory associated with a forensic investigation or include forensic samples obtained by law enforcement agencies, one or more military services or any such personnel. The nucleic acid sample may be a purified sample or a crude DNA containing lysate, for example, derived from a buccal swab, paper, fabric, or other substrates that may be impregnated with saliva, blood, or other bodily fluids. As such, in some implementations, the nucleic acid sample may comprise low amounts of, or fragmented portions of DNA, such as genomic DNA. In some implementations, target sequences can be present in one or more bodily fluids including but not limited to, blood, sputum, plasma, semen, urine, and serum. In some implementations, target sequences can be obtained from hair, skin, tissue samples, autopsy or remains of a victim. In some implementations, nucleic acids including one or more target sequences can be obtained from a deceased animal or human. In some implementations, target sequences can include nucleic acids obtained from non-human DNA such a microbial, plant, or entomological DNA. In some implementations, target sequences or amplified target sequences are directed to purposes of human identification. In some implementations, the disclosure relates generally to methods for identifying characteristics of a forensic sample. In some implementations, the disclosure relates generally to human identification methods using one or more target-specific primers disclosed herein or one or more target-specific primers designed using the primer design criteria outlined herein. In one implementation, a forensic or human identification sample containing at least one target sequence can be amplified using any one or more of the target-specific primers disclosed herein or using the primer criteria outlined herein.

The components of the MRD detection system 106 can include software, hardware, or both. For example, the components of the MRD detection system 106 can include one or more instructions stored on a computer-readable storage medium and executable by processors of one or more computing devices (e.g., the server device(s) 102, the client device 110, and/or the sequencing device 114). When executed by the one or more processors, the computer-executable instructions of the MRD detection system 106 can cause the computing devices to perform the MRD detection process described herein. Alternatively, the components of the MRD detection system 106 can comprise hardware, such as special purpose processing devices to perform a certain function or group of functions. Additionally, or alternatively, the components of the MRD detection system 106 can include a combination of computer-executable instructions and hardware.

Furthermore, the components of the MRD detection system 106 performing the functions described herein with respect to the MRD detection system 106 may, for example, be implemented as part of a stand-alone application, as a module of an application, as a plug-in for applications, as a library function or functions that may be called by other applications, and/or as a cloud-computing model. Thus, components of the MRD detection system 106 may be implemented as part of a stand-alone application on a personal computing device or a mobile device. Additionally, or alternatively, the components of the MRD detection system 106 may be implemented in any application that provides sequencing services including, but not limited to Illumina BaseSpace, Illumina DRAGEN, or Illumina TruSight software. “Illumina,” “BaseSpace,” “DRAGEN,” and “TruSight,” are either registered trademarks or trademarks of Illumina, Inc. in the United States and/or other countries.

Embodiments of the present disclosure may comprise or utilize a special purpose or general-purpose computer including computer hardware, such as, for example, one or more processors and system memory, as discussed in greater detail below. Embodiments within the scope of the present disclosure also include physical and other computer-readable media for carrying or storing computer-executable instructions and/or data structures. In particular, one or more of the processes described herein may be implemented at least in part as instructions embodied in a non-transitory computer-readable medium and executable by one or more computing devices (e.g., any of the media content access devices described herein). In general, a processor (e.g., a microprocessor) receives instructions, from a non-transitory computer-readable medium, (e.g., a memory, etc.), and executes those instructions, thereby performing one or more processes, including one or more of the processes described herein.

Computer-readable media can be any available media that can be accessed by a general purpose or special purpose computer system. Computer-readable media that store computer-executable instructions are non-transitory computer-readable storage media (devices). Computer-readable media that carry computer-executable instructions are transmission media. Thus, by way of example, and not limitation, embodiments of the disclosure can comprise at least two distinctly different kinds of computer-readable media: non-transitory computer-readable storage media (devices) and transmission media.

Non-transitory computer-readable storage media (devices) includes RAM, ROM, EEPROM, CD-ROM, solid state drives (SSDs) (e.g., based on RAM), Flash memory, phase-change memory (PCM), other types of memory, other optical disk storage, magnetic disk storage or other magnetic storage devices, or any other medium which can be used to store desired program code means in the form of computer-executable instructions or data structures and which can be accessed by a general purpose or special purpose computer.

A “network” is defined as one or more data links that enable the transport of electronic data between computer systems and/or modules and/or other electronic devices. When information is transferred or provided over a network or another communications connection (either hardwired, wireless, or a combination of hardwired or wireless) to a computer, the computer properly views the connection as a transmission medium. Transmissions media can include a network and/or data links which can be used to carry desired program code means in the form of computer-executable instructions or data structures and which can be accessed by a general purpose or special purpose computer. Combinations of the above should also be included within the scope of computer-readable media.

Further, upon reaching various computer system components, program code means in the form of computer-executable instructions or data structures can be transferred automatically from transmission media to non-transitory computer-readable storage media (devices) (or vice versa). For example, computer-executable instructions or data structures received over a network or data link can be buffered in RAM within a network interface module (e.g., a NIC), and then eventually transferred to computer system RAM and/or to less volatile computer storage media (devices) at a computer system. Thus, it should be understood that non-transitory computer-readable storage media (devices) can be included in computer system components that also (or even primarily) utilize transmission media.

Computer-executable instructions comprise, for example, instructions and data which, when executed at a processor, cause a general-purpose computer, special purpose computer, or special purpose processing device to perform a certain function or group of functions. In some embodiments, computer-executable instructions are executed on a general-purpose computer to turn the general-purpose computer into a special purpose computer implementing elements of the disclosure. The computer executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the disclosure may be practiced in network computing environments with many types of computer system configurations, including, personal computers, desktop computers, laptop computers, message processors, hand-held devices, multi-processor systems, microprocessor-based or programmable consumer electronics, network PCs, minicomputers, mainframe computers, mobile telephones, PDAs, tablets, pagers, routers, switches, and the like. The disclosure may also be practiced in distributed system environments where local and remote computer systems, which are linked (either by hardwired data links, wireless data links, or by a combination of hardwired and wireless data links) through a network, both perform tasks. In a distributed system environment, program modules may be located in both local and remote memory storage devices.

Embodiments of the present disclosure can also be implemented in cloud computing environments. In this description, “cloud computing” is defined as a model for enabling on-demand network access to a shared pool of configurable computing resources. For example, cloud computing can be employed in the marketplace to offer ubiquitous and convenient on-demand access to the shared pool of configurable computing resources. The shared pool of configurable computing resources can be rapidly provisioned via virtualization and released with low management effort or service provider interaction, and then scaled accordingly.

A cloud-computing model can be composed of various characteristics such as, for example, on-demand self-service, broad network access, resource pooling, rapid elasticity, measured service, and so forth. A cloud-computing model can also expose various service models, such as, for example, Software as a Service (SaaS), Platform as a Service (PaaS), and Infrastructure as a Service (IaaS). A cloud-computing model can also be deployed using different deployment models such as private cloud, community cloud, public cloud, hybrid cloud, and so forth. In this description and in the claims, a “cloud-computing environment” is an environment in which cloud computing is employed.

FIG. 10 illustrates a block diagram of a computing device 1000 that may be configured to perform one or more of the processes described above. One will appreciate that one or more computing devices such as the computing device 1000 may implement the MRD detection system 106. As shown by FIG. 10, the computing device 1000 can comprise a processor 1002, a memory 1004, a storage device 1006, an I/O interface 1008, and a communication interface 1010, which may be communicatively coupled by way of a communication infrastructure 1012. In certain embodiments, the computing device 1000 can include fewer or more components than those shown in FIG. 10. The following paragraphs describe components of the computing device 1000 shown in FIG. 10 in additional detail.

In one or more embodiments, the processor 1002 includes hardware for executing instructions, such as those making up a computer program. As an example, and not by way of limitation, to execute instructions for dynamically modifying workflows, the processor 1002 may retrieve (or fetch) the instructions from an internal register, an internal cache, the memory 1004, or the storage device 1006 and decode and execute them. The memory 1004 may be a volatile or non-volatile memory used for storing data, metadata, and programs for execution by the processor(s). The storage device 1006 includes storage, such as a hard disk, flash disk drive, or other digital storage device, for storing data or instructions for performing the methods described herein.

The I/O interface 1008 allows a user to provide input to, receive output from, and otherwise transfer data to and receive data from computing device 1000. The I/O interface 1008 may include a mouse, a keypad or a keyboard, a touch screen, a camera, an optical scanner, network interface, modem, other known I/O devices or a combination of such I/O interfaces. The I/O interface 1008 may include one or more devices for presenting output to a user, including, but not limited to, a graphics engine, a display (e.g., a display screen), one or more output drivers (e.g., display drivers), one or more audio speakers, and one or more audio drivers. In certain embodiments, the I/O interface 1008 is configured to provide graphical data to a display for presentation to a user. The graphical data may be representative of one or more graphical user interfaces and/or any other graphical content as may serve a particular implementation.

The communication interface 1010 can include hardware, software, or both. In any event, the communication interface 1010 can provide one or more interfaces for communication (such as, for example, packet-based communication) between the computing device 1000 and one or more other computing devices or networks. As an example, and not by way of limitation, the communication interface 1010 may include a network interface controller (NIC) or network adapter for communicating with an Ethernet or other wire-based network or a wireless NIC (WNIC) or wireless adapter for communicating with a wireless network, such as a WI-FI.

Additionally, the communication interface 1010 may facilitate communications with various types of wired or wireless networks. The communication interface 1010 may also facilitate communications using various communication protocols. The communication infrastructure 1012 may also include hardware, software, or both that couples components of the computing device 1000 to each other. For example, the communication interface 1010 may use one or more networks and/or protocols to enable a plurality of computing devices connected by a particular infrastructure to communicate with each other to perform one or more aspects of the processes described herein. To illustrate, the sequencing process can allow a plurality of devices (e.g., a client device, sequencing device, and server device(s)) to exchange information such as sequencing data and error notifications.

In the foregoing specification, the present disclosure has been described with reference to specific exemplary embodiments thereof. Various embodiments and aspects of the present disclosure(s) are described with reference to details discussed herein, and the accompanying drawings illustrate the various embodiments. The description above and drawings are illustrative of the disclosure and are not to be construed as limiting the disclosure. Numerous specific details are described to provide a thorough understanding of various embodiments of the present disclosure.

The present disclosure may be embodied in other specific forms without departing from its spirit or essential characteristics. The described embodiments are to be considered in all respects only as illustrative and not restrictive. For example, the methods described herein may be performed with less or more steps/acts or the steps/acts may be performed in differing orders. Additionally, the steps/acts described herein may be repeated or performed in parallel with one another or in parallel with different instances of the same or similar steps/acts. The scope of the present application is, therefore, indicated by the appended claims rather than by the foregoing description. All changes that come within the meaning and range of equivalency of the claims are to be embraced within their scope.

What is claimed is:

1. A system comprising:
at least one processor; and
a non-transitory computer-readable medium storing instructions that, when executed by the at least one processor, cause the system to:
identify, for an initial genomic sample of a subject infected with a type of cancer, a tumor fingerprint comprising variants at a target genomic region;
determine, for a sample of interest corresponding to a subsequent genomic sample of the subject, a set of sample of interest nucleotide reads associated with the target genomic region;
process, using a first machine learning model that was trained with sample of interest training data, extracted features from the set of sample of interest nucleotide reads to generate a sample of interest score;
process, using one or more additional machine learning models that were trained with panel of normals training data, extracted features from panel of normals nucleotide reads associated with the target genomic region to generate one or more panel of normals scores; and
compare the sample of interest score to the one or more panel of normals scores to predict whether the sample of interest has minimal residual disease related to the type of cancer.

2. The system of claim 1, wherein the sample of interest training data used to train the first machine learning model comprises:
a class 1 dataset corresponding to tumor supporting nucleotide reads; and
a first class 0 dataset corresponding to non-tumor nucleotide reads from pseudo fingerprint genomic regions within the sample of interest.

3. The system of claim 2, wherein the pseudo fingerprint genomic regions within the sample of interest comprise regions that do not overlap the target genomic region associated with the tumor fingerprint.

4. The system of claim 3, wherein the panel of normals training data used to train the one or more additional machine learning models comprises:
the class 1 dataset corresponding to the tumor supporting nucleotide reads; and
one or more additional class 0 datasets corresponding to one or more samples within a panel of normals, where each of the one or more additional machine learning models corresponds to a specific sample from the one or more samples within the panel of normals.

5. The system of claim 2, wherein the tumor supporting nucleotide reads of the class 1 dataset includes a plurality of paired-end reads corresponding to a plurality of tumor samples, wherein each paired-end read corresponds to a tumor sample and comprises a first read and a second read that overlap with a genomic region of a fingerprint for the tumor sample and support a tumor allele associated with tumor sample.

6. The system of claim 1, wherein the initial genomic sample comprises a sample from a tumor of the subject and the subsequent genomic sample comprises a plasma sample comprising cell-free deoxyribonucleic acid (cfDNA).

7. The system of claim 1, further comprising instructions that, when executed by the at least one processor, cause the system to extract features from the set of sample of interest nucleotide reads associated with the target genomic region.

8. The system of claim 7, wherein extracting the features from the set of sample of interest nucleotide reads associated with the target genomic region comprises extracting the features from a plurality of paired-end reads, each paired-end read comprising a first read and a second read that overlap with the target genomic region of the tumor fingerprint.

9. The system of claim 8, wherein the extracted features comprise at least one of:
individual read features for the first read of each of the plurality of paired-end reads;
individual read features for the second read of each of the plurality of paired-end reads; or
combination read features corresponding to properties associated with the combination of the first read and the second read.

10. A non-transitory computer-readable medium storing instructions that, when executed by at least one processor, cause a computing device to:
identify, for an initial genomic sample of a subject infected with a type of cancer, a tumor fingerprint comprising variants at a target genomic region;
determine, for a sample of interest corresponding to a subsequent genomic sample of the subject, a set of sample of interest nucleotide reads associated with the target genomic region;
process, using a first machine learning model that was trained with sample of interest training data, extracted features from the set of sample of interest nucleotide reads to generate a sample of interest score;
process, using one or more additional machine learning models that were trained with panel of normals training data, extracted features from panel of normals nucleotide reads associated with the target genomic region to generate one or more panel of normals scores; and
compare the sample of interest score to the one or more panel of normals scores to predict whether the sample of interest has minimal residual disease related to the type of cancer.

11. The non-transitory computer-readable medium of claim 10, wherein the sample of interest training data used to train the first machine learning model comprises:
a class 1 dataset corresponding to tumor supporting nucleotide reads; and
a first class 0 dataset corresponding to non-tumor nucleotide reads from pseudo fingerprint genomic regions within the sample of interest.

12. The non-transitory computer-readable medium of claim 11, wherein the pseudo fingerprint genomic regions within the sample of interest comprise regions that do not overlap the target genomic region associated with the tumor fingerprint.

13. The non-transitory computer-readable medium of claim 12, wherein the panel of normals training data used to train the one or more additional machine learning models comprises:
the class 1 dataset corresponding to the tumor supporting nucleotide reads; and
one or more additional class 0 datasets corresponding to one or more samples within a panel of normals, where each of the one or more additional machine learning models corresponds to a specific sample from the one or more samples within the panel of normals.

14. The non-transitory computer-readable medium of claim 11, wherein the tumor supporting nucleotide reads of the class 1 dataset includes a plurality of paired-end reads corresponding to a plurality of tumor samples, wherein each paired-end read corresponds to a tumor sample and comprises a first read and a second read that overlap with a genomic region of a fingerprint for the tumor sample and support a tumor allele associated with tumor sample.

15. The non-transitory computer-readable medium of claim 10, wherein the initial genomic sample comprises a sample from a tumor of the subject and the subsequent genomic sample comprises a plasma sample comprising cell-free deoxyribonucleic acid (cfDNA).

16. The non-transitory computer-readable medium of claim 10, further comprising instructions that, when executed by the at least one processor, cause the computing device to extract features from the set of sample of interest nucleotide reads associated with the target genomic region.

17. The non-transitory computer-readable medium of claim 16, wherein extracting the features from the set of sample of interest nucleotide reads associated with the target genomic region comprises extracting the features from a plurality of paired-end reads, each paired-end read comprising a first read and a second read that overlap with the target genomic region of the tumor fingerprint.

18. The non-transitory computer-readable medium of claim 17, wherein the extracted features comprise at least one of:
individual read features for the first read of each of the plurality of paired-end reads;
individual read features for the second read of each of the plurality of paired-end reads; or
combination read features corresponding to properties associated with the combination of the first read and the second read.

19. A method comprising:
identifying, for an initial genomic sample of a subject infected with a type of cancer, a tumor fingerprint comprising variants at a target genomic region;
determining, for a sample of interest corresponding to a subsequent genomic sample of the subject, a set of sample of interest nucleotide reads associated with the target genomic region;
processing, using a first machine learning model that was trained with sample of interest training data, extracted features from the set of sample of interest nucleotide reads to generate a sample of interest score;
processing, using one or more additional machine learning models that were trained with panel of normals training data, extracted features from panel of normals nucleotide reads associated with the target genomic region to generate one or more panel of normals scores; and
comparing the sample of interest score to the one or more panel of normals scores to predict whether the sample of interest has minimal residual disease related to the type of cancer.

20. The method of claim 19, wherein the sample of interest training data used to train the first machine learning model comprises:
a class 1 dataset corresponding to tumor supporting nucleotide reads; and
a first class 0 dataset corresponding to non-tumor nucleotide reads from pseudo fingerprint genomic regions within the sample of interest.
